SEQUENCE LISTING (21 sequences)

SEQ ID NO: 1 - DNA, 930 bp, Arabidopsis thaliana
atggagaagtacgagaagctagagaaggtcggagaaggaacatacgggaaagtctacaaagcgatggagaaaggaactggtaagcttgttgctctgaagaaaactcgtctcgagatggacgaagaaggtattccaccaactgctcttcgtgagatctcgcttctccagatgttatcaacatcgatctatgttgttcgattactctgcgtcgaacatgttcatcaaccatcaaccaaatctcaatctaccaaatccaatctctatctcgttttcgagtatctcgatactgatcttaagaaattcatcgattcgtataggaaaggacctaatcctaagcctcttgagccttttttgattcagaagttgatgtttcagctttgtaaaggtgttgcgcattgtcatagtcatggtgtgcttcaccgtgatcttaaaccgcagaatcttcttctggtgaaagataaagagcttcttaagattgctgatttgggtcttggtcgtgcttttactgttcctcttaagtcttatacgcatgagattgttactctttggtatagagctcctgaagttcttcttggatctactcattattcaactggtgttgacatgtggtctgttggttgtatctttgctgagatggttcggaggcaagctcttttccctggtgattctgagtttcagcaattgcttcatatcttcaggttgctaggaacaccaactgagcagcaatggccgggtgtttccacactgcgtgactggcatgtttaccctaagtgggagccgcaagacttaactcttgctgttccttctctttcacctcaaggagttgatcttctcacgaaaatgctcaagtacaatccagccgaaagaatttcagcaaaaacagcacttgatcacccatattttgacagccttgacaagtctcagttctga

SEQ ID NO: 2 - protein, 309 aa, Arabidopsis thaliana
MEKYEKLEKVGEGTYGKVYKAMEKGTGKLVALKKTRLEMDEEGIPPTALREISLLQMLSTSIYVVRLLCVEHVHQPSTKSQSTKSNLYLVFEYLDTDLKKFIDSYRKGPNPKPLEPFLIQKLMFQLCKGVAHCHSHGVLHRDLKPQNLLLVKDKELLKIADLGLGRAFTVPLKSYTHEIVTLWYRAPEVLLGSTHYSTGVDMWSVGCIFAEMVRRQALFPGDSEFQQLLHIFRLLGTPTEQQWPGVSTLRDWHVYPKWEPQDLTLAVPSLSPQGVDLLTKMLKYNPAERISAKTALDHPYFDSLDKSQF

SEQ ID NO: 3 - DNA, 936 bp, Arabidopsis thaliana
atggagaaatacgagaagctcgaaaaggtcggtgaaggaacctatggaaaagtctacaaagcaatggagaaaaccaccggaaaactcgtcgctctgaagaaaactaggctcgaaatggacgaagaaggtataccaccaacggctctccgtgagatctctcttctccaaatgctttctcaatcaatctacatcgttcgtctcctctgcgtcgaacatgttattcaatcgaaagattcgactgtttctcactctcccaaatccaatctctatctcgtttttgagtatctcgacactgatctcaagaaatttatagattctcatagaaagggctcgaatcctagaccgcttgaggcttctcttgtgcagaggtttatgtttcagctttttaaaggtgtggctcattgtcatagccatggtgtgcttcaccgtgatcttaaaccgcagaatcttctattggataaggataaagggattcttaagattgctgatttgggtcttagtcgtgcttttactgtgcctcttaaggcttatacacatgagattgttactctttggtatagagctcctgaagttttgcttggttctactcattactctactgctgttgatatttggtctgttggatgcatctttgccgagatgattaggaggcaagctcttttccctggtgattctgagtttcagcaactacttcatattttcagattgttaggaacaccaactgagcagcaatggccgggtgtaatggcattgcgtgactggcatgtctatccaaagtgggagccgcaagacttatcacgtgctgttccatctctatctcctgaaggaattgatcttctcacgcaaatgttgaagtacaatccagcagaaagaatttcagcaaaagcagctctcgatcatccctactttgacagccttgacaaatctcagttctga

SEQ ID NO: 4 - protein, 311 aa, Arabidopsis thaliana
MEKYEKLEKVGEGTYGKVYKAMEKTTGKLVALKKTRLEMDEEGIPPTALREISLLQMLSQSIYIVRLLCVEHVIQSKDSTVSHSPKSNLYLVFEYLDTDLKKFIDSHRKGSNPRPLEASLVQRFMFQLFKGVAHCHSHGVLHRDLKPQNLLLDKDKGILKIADLGLSRAFTVPLKAYTHEIVTLWYRAPEVLLGSTHYSTAVDIWSVGCIFAEMIRRQALFPGDSEFQQLLHIFRLLGTPTEQQWPGVMALRDWHVYPKWEPQDLSRAVPSLSPEGIDLLTQMLKYNPAERISAKAALDHPYFDSLDKSQF

SEQ ID NO: 5 - DNA, 948 bp, Arabidopsis thaliana
atggacaacaatggagttaaacccgctgtttccgccatggaagcctttgaaaagcttgagaaagtaggtgaagggacttatgggaaagtttacagagcaagagagaaagctactgggatgatcgttgctttgaagaagacgcgtctccatgaggatgaagaaggtgttcctcccactactcttcgcgagatctctatcttgcgtatgctcgctcgtgatcctcacatcgttaggttgatggatgttaagcaaggaataaacaaagaaggaaaaactgtactttaccttgttttcgagtatgttgatactgatctcaagaaattcatcagaagctttcgtcaagctggacagaacattccacaaaatactgtcaagtgcttgatgtaccagttatgcaaaggcatggctttttgccatggtcatggagtgttgcacagggatcttaagcctcacaatctcttgatggaccggaagacaatgacgctcaaaatagcagatcttggattagccagagccttcactctcccaatgaaaaagtatacacatgagattctaactctatggtatagagctccggaagttcttcttggagcaacccattactctactggagtggatatgtggtctgttggctgtatttttgctgaactagtgaccaagcaagcaatctttgcgggagactctgagctccaacagctcctccgtatattcaggttgttgggaacaccaaacgaagaagtttggcctggagtaagcaaactcaaggactggcatgaatacccgcaatggaaaccgttgagtctctccacagctgtgccaaacctcgacgaggctggacttgatctcttatctaaaatgctggagtacgagccagcaaaacgaatctcagcaaagaaagctatggagcatccttacttcgatgatttgcctgacaagtcctctctctga

SEQ ID NO: 6 - protein, 315 aa, Arabidopsis thaliana
MDNNGVKPAVSAMEAFEKLEKVGEGTYGKVYRAREKATGMIVALKKTRLHEDEEGVPPTTLREISILRMLARDPHIVRLMDVKQGINKEGKTVLYLVFEYVDTDLKKFIRSFRQAGQNIPQNTVKCLMYQLCKGMAFCHGHGVLHRDLKPHNLLMDRKTMTLKIADLGLARAFTLPMKKYTHEILTLWYRAPEVLLGATHYSTGVDMWSVGCIFAELVTKQAIFAGDSELQQLLRIFRLLGTPNEEVWPGVSKLKDWHEYPQWKPLSLSTAVPNLDEAGLDLLSKMLEYEPAKRISAKKAMEHPYFDDLPDKSSL

SEQ ID NO: 7 - DNA, 1115 bp, Oryza sativa
acctctcctccgattaatcccctcccctcctcttcctcccacttctgcgcctgctcttcctcccctcgccgaccctacctactcgcgccgccgccgtcgcattgggcggcaaacggagggggggttaaccctgatggagcagtacgagaaggaggagaagattggggagggcacgtacggggtggtgtacagggcgcgggacaaggtcaccaacgagacgatcgcgctcaagaagatccggcttgagcaggaggatgagggcgtcccctccaccgcaatccgcgagatctcgctcctcaaggagatgcatcacggcaacatcgtcaggttacacgatgttatccacagtgagaagcgcatatatcttgtctttgagtatctggatctggacctaaagaagttcatggactcttgtccagagtttgcgaaaaaccccactttaattaagtcatatctctatcagatactccgcggcgttgcttactgtcattctcatagagttcttcatcgagatttgaaacctcagaatttattgatagatcggcgtactaatgcactgaagcttgcagactttggtttagccagggcatttggaattcctgtccgcacgtttactcacgaggttgtaaccttgtggtatagagctccagagatccttcttggatcaaggcagtattctacaccagttgatatgtggtcagttggttgtatctttgcagaaatggtgaaccagaaaccactgttccctggtgattctgagattgatgaattatttaagatattcagggtactaggaactccaaatgaacaaagttggccaggagttagctcattacctgactacaagtctgctttccccaagtggcaagcacaggatcttgcaactattgtccctactcttgaccctgctggtttggaccttctctctaaaatgcttcggtacgagccaaacaaaaggatcacagctagacaggctcttgagcatgaatacttcaaggaccttgagatggtacaatgaccctgctatggctttacattggattggcatatgtatgggctgggctcctcatttcattccttctgtgaacgctgtgcccttcgtttgggcatttttg

SEQ ID NO: 8 - protein, 294 aa, Oryza sativa
MEQYEKEEKIGEGTYGVVYRARDKVTNETIALKKIRLEQEDEGVPSTAIREISLLKEMHHGNIVRLHDVIHSEKRIYLVFEYLDLDLKKFMDSCPEFAKNPTLIKSYLYQILRGVAYCHSHRVLHRDLKPQNLLIDRRTNALKLADFGLARAFGIPVRTFTHEVVTLWYRAPEILLGSRQYSTPVDMWSVGCIFAEMVNQKPLFPGDSEIDELFKIFRVLGTPNEQSWPGVSSLPDYKSAFPKWQAQDLATIVPTLDPAGLDLLSKMLRYEPNKRITARQALEHEYFKDLEMVQ

SEQ ID NO: 9 - protein, 294 aa, Oryza sativa
MEQHEKEEKIGEGTYGVVYRARDKVTNETIALKKIRLEQEDEGVPSTAIREISLLKEMHHGNIVRLHDVIHSEKRIYLDFEYLDLDLKKFMDSCPEFAKNPTLIKSYLYQILRGVAYCHSHRVLHRDLKPQNLLIDRRTNALKLADFGLARTFGIPVRTFTHEVVTLWYRAPEILLGSRQYSTPVDMWSVGCIFAEMVNQKPLFPGDSEIDELFKIFRVLGTPNEQSWPGVSSLPDYKSAFPKWQAQDLATIVPTLDPAGLDLLSKMLRYEPNKRITARQALEHEYFKDLEMVQ

SEQ ID NO: 10 - protein, 294 aa, Oryza sativa
MEQYEKEEKIGEGTYGVVYRARDKVTNETTALKKIRLEQEDEGVPSTAIREISLLKEMHHGNIVRLHDVIHSEKRIYLVFEYLDLDLKKFMDSCPEFAKNPTLIKSYLYQILRGVAYCHSHRVLHRDLKPQNLLIDRRTNALKLADFGLARAFGIPVRTFTHEVVTLWYRAPEILLGSRQYSTPVDMWSVGCIFAEMVNQKPLFPGDSEIDELFKIFRVLGTPNEQSWPGVSSLPDYKSAFPKWQAQDLATIVPTLDPAGLDLLSKMLRYEPNKRITARQALEHEYFKDLEMVQ

SEQ ID NO: 11 - protein, 294 aa, Oryza sativa
MEQYVKEEKIGEGTYGVVYRARDKVTNETIALKKIRLEQEDEGVPSTAIREISLLKEMHHGNIVRLHDVIHSEKRIYLVFEYLDLDLKKFMDSCPEFAKNPTLIKSYLYQILRGVAYCHSHSVLHRDLKPQNLLIDRRTNALELADFGLARAFGIPVRTFTHEVVTLWYRAPEILLGSRQYSTPVDMWSVGCIFAEMVNQKPLFPGDSEIDELFKIFRVLGTPNEQSWPGVSSLPDYKSAFPKWQAQDLATIVPTLDPAGLDLLSKMLRYEPNKRITARQALEHEYFKDLEMVQ

SEQ ID NO: 12 - protein, 294 aa, Oryza sativa
MEQYEKEEKIGEGTYGVVYRARDKVTNETIALKKIRLEQEDEGVPSTAIREISLLKEMHHGNIVRLHDVIHSEKRIYLVFEYLDLDLKKFMDSCPEFAKNPTLIKSYLYQILRGVAYCHSHRVLHRDLKPQNLLIDRRTNALKLADFGLARAFRIPVRTFTHEVVTLWYRAPEILLGSRQYSTPVDMWSVGCIFAEMVNQKPLFPGDSEIDELFKIFRVLGTPNEQSWPGVSSLPDYKSAFPKWQAQDLATIVPTLDPAGLDLLSKMLRYEPNKRITARQALEHEYFKDLEMVQ

SEQ ID NO: 13 - protein, 294 aa, Oryza sativa
MEPYEKEEKIGEGTYGVVYRARDKVTNETIALKKIRLAQEDEGVPSTAIREISLLKEMHHGNIVRLHDVIHSEKRIYLVFEYLDLDLKKFMDSCPEFAKNPTLIKSYLYQILRGVAYCHSHRVLHRDLKPQNLLIDLRTNALKLADFGLARAFGIPVRTFTHEVVTLWYRAPEILLGSRQYATPVDMWSVGCTFAEMVNQKPLFPGDSEIDELFKIFRVLGTPNEQSWPGVSSLPDYKSAFPKWQAQDLATIVPTLDPAGLDLLSKVLRYEPNKRITAQQALEHEYFKDLEMVQ

SEQ ID NO: 14 - DNA, 1243 bp, Oryza sativa
aaaaccaccgagggacctgatctgcaccggttttgatagttgagggacccgttgtgtctggttttccgatcgagggacgaaaatcggattcggtgtaaagttaagggacctcagatgaacttattccggagcatgattgggaagggaggacataaggcccatgtcgcatgtgtttggacggtccagatctccagatcactcagcaggatcggccgcgttcgcgtagcacccgcggtttgattcggcttcccgcaaggcggcggccggtggccgtgccgccgtagcttccgccggaagcgagcacgccgccgccgccgacccggctctgcgtttgcaccgccttgcacgcgatacatcgggatagatagctactactctctccgtttcacaatgtaaatcattctactattttccacattcatattgatgttaatgaatatagacatatatatctatttagattcattaacatcaatatgaatgtaggaaatgctagaatgacttacattgtgaattgtgaaatggacgaagtacctacgatggatggatgcaggatcatgaaagaattaatgcaagatcgtatctgccgcatgcaaaatcttactaattgcgctgcatatatgcatgacagcctgcatgcgggcgtgtaagcgtgttcatccattaggaagtaaccttgtcattacttataccagtactacatactatatagtattgatttcatgagcaaatctacaaaactggaaagcaataagaaatacgggactggaaaagactcaacattaatcaccaaatatttcgccttctccagcagaatatatatctctccatcttgatcactgtacacactgacagtgtacgcataaacgcagcagccagcttaactgtcgtctcaccgtcgcacactggccttccatctcaggctagctttctcagccacccatcgtacatgtcaactcggcgcgcgcacaggcacaaattacgtacaaaacgcatgaccaaatcaaaaccaccggagaagaatcgctcccgcgcgcggcggcgacgcgcacgtacgaacgcacgcacgcacgcccaaccccacgacacgatcgcgcgcgacgccggcgacaccggccgtccacccgcgccctcacctcgccgactataaatacgtaggcatctgcttgatcttgtcatccatctcaccaccaaaaaaaaaaggaaaaaaaaacaaaacacaccaagccaaataaaagcgaca

SEQ ID NO: 15 - DNA, 2191 bp, Oryza sativa
aatccgaaaagtttctgcaccgttttcaccccctaactaacaatatagggaacgtgtgctaaatataaaatgagaccttatatatgtagcgctgataactagaactatgcaagaaaaactcatccacctactttagtggcaatcgggctaaataaaaaagagtcgctacactagtttcgttttccttagtaattaagtgggaaaatgaaatcattattgcttagaatatacgttcacatctctgtcatgaagttaaattattcgaggtagccataattgtcatcaaactcttcttgaataaaaaaatctttctagctgaactcaatgggtaaagagagagattttttttaaaaaaatagaatgaagatattctgaacgtattggcaaagatttaaacatataattatataattttatagtttgtgcattcgtcatatcgcacatcattaaggacatgtcttactccatcccaatttttatttagtaattaaagacaattgacttatttttattatttatcttttttcgattagatgcaaggtacttacgcacacactttgtgctcatgtgcatgtgtgagtgcacctcctcaatacacgttcaactagcaacacatctctaatatcactcgcctatttaatacatttaggtagcaatatctgaattcaagcactccaccatcaccagaccacttttaataatatctaaaatacaaaaaataattttacagaatagcatgaaaagtatgaaacgaactatttaggtttttcacatacaaaaaaaaaaagaattttgctcgtgcgcgagcgccaatctcccatattgggcacacaggcaacaacagagtggctgcccacagaacaacccacaaaaaacgatgatctaacggaggacagcaagtccgcaacaaccttttaacagcaggctttgcggccaggagagaggaggagaggcaaagaaaaccaagcatcctcctcctcccatctataaattcctccccccttttcccctctctatataggaggcatccaagccaagaagagggagagcaccaaggacacgcgactagcagaagccgagcgaccgccttcttcgatccatatcttccggtcgagttcttggtcgatctcttccctcctccacctcctcctcacagggtatgtgcccttcggttgttcttggatttattgttctaggttgtgtagtacgggcgttgatgttaggaaaggggatctgtatctgtgatgattcctgttcttggatttgggatagaggggttcttgatgttgcatgttatcggttcggtttgattagtagtatggttttcaatcgtctggagagctctatggaaatgaaatggtttagggtacggaatcttgcgattttgtgataccttttgtttgaggtaaaatcagagcaccggtgattttgcttggtgtaataaaagtacggttgtttggtcctcgattctggtagtgatgcttctcgatttgacgaagctatcctttgtttattccctattgaacaaaaataatccaactttgaagacggtcccgttgatgagattgaatgattgattcttaagcctgtccaaaatttcgcagctggcttgtttagatacagtagtccccatcacgaaattcatggaaacagttataatcctcaggaacaggggattccctgttcttccgatttgctttagtcccagaattttttttcccaaatatcttaaaaagtcactttctggttcagttcaatgaattgattgctacaaataatgcttttatagcgttatcctagctgtagttcagttaataggtaatacccctatagtttagtcaggagaagaacttatccgatttctgatctccatttttaattatatgaaatgaactgtagcataagcagtattcatttggattattttttttttagctctcaccccttcattattctgagctgaaagtctggcatgaactgtcctcaattttgttttcaaattcacatcgattatctatgcattatcctcttgtatctacctgtagaagtttctttttggttattccttgactgcttgattacagaaagaaatttatgaagctgtaatcgggatagttatactgcttgttcttatgattcatttcctttgtgcagttcttggtgtagcttgccactttcaccagcaaagttc

SEQ ID NO: 16 - DNA, 57 bp, Artificial sequence
  sense primer prm0350
ggggacaagtttgtacaaaaaagcaggcttcacaatggagaagtacgagaagctaga

SEQ ID NO: 17 - DNA, 51 bp, Artificial sequence
  antisense primer prm0351
ggggaccactttgtacaagaaagctgggttcagaactgagacttgtcaagg

SEQ ID NO: 18 - DNA, 55 bp, Artificial sequence
  sense primer prm439
ggggacaagtttgtacaaaaaagcaggcttcacaatggagaaatacgagaagctc

SEQ ID NO: 19 - DNA, 49 bp, Artificial sequence
  antisense primer prm440
ggggaccactttgtacaagaaagctgggtggtcagaactgagatttgtc

SEQ ID NO: 20 - DNA, 54 bp, Artificial sequence
  sense primer prm2213
ggggacaagtttgtacaaaaaagcaggcttcacaatggacaacaatggagttaa

SEQ ID NO: 21 - DNA, 49 bp, Artificial sequence
  antisense primer prm2214
ggggaccactttgtacaagaaagctgggttcagagagaggacttgtcag

(19) United States
(12) Patent Application Publication
(10) Pub. No.: US 2007/0199085 A1
(43) Pub. Date: Aug. 23, 2007

(54) Plants Having Modified Growth Characteristics And Method For Making Same

(76) Inventors: Willem Broekaert, Dilbeek (BE); Valerie Frankard, Sint-Genesius-Rode (BE); Yves Hatzfeld, Lille (FR); Vladimir Mironov, Gent (BE)

Correspondence Address: DILWORTH & BARRESE, LLP, 333 EARLE OVINGTON BLVD., SUITE 702, UNIONDALE, NY, 11553, US

(21) Appl. No.: 10/570,554

(22) PCT Filed: Sep. 3, 2004
(86) PCT No.: PCT/EP04/52035

§ 371 Date: Dec. 7, 2006

(30) Foreign Application Priority Data
Sep. 5, 2003 (EP) 03077811.2

(51) Int. Cl. A01H 1/00; C07H 21/04; C12N 9/12; C12N 15/82; C12N 5/04

(52) U.S. Cl. 800/278; 435/419; 435/468; 536/23.6; 435/194

(57) ABSTRACT

The present invention concerns a method for modifying the growth characteristics of plants by modulating expression in a plant of a B-type CDK nucleic acid and/or modulating activity and/or levels in a plant of a B-type CDK protein. The invention also relates to transgenic plants having modified growth characteristics, which plants have modulated expression of a B-type CDK nucleic acid and/or modulated activity and/or levels in a plant of a B-type CDK protein relative to expression, activity and/or levels in corresponding wild-type plants. The present invention also provides a novel screening method for the identification of mutant CDKs having enhanced CDK activity relative to corresponding non-mutated CDKs. The present invention also provides a novel screening method for the identification of non-active CDKs that are able to bind to CKIs (cyclin dependent kinase inhibitors). The invention also provides mutant CDKs obtainable by the screening methods according to the invention.

The present invention relates generally to the field of molecular biology and concerns a method for modifying plant growth characteristics. More specifically, the present invention concerns a method for modifying plant growth characteristics by modulating expression in a plant of a B-type CDK (cyclin dependent kinase) nucleic acid and/or by modulating activity and/or levels in a plant of a B-type CDK protein. The present invention also concerns plants having modulated expression of a B-type CDK nucleic acid and/or modulated activity and/or levels of a B-type CDK protein, which plants have modified growth characteristics relative to corresponding wild type plants. The present invention also provides a novel screening method for the identification of mutant CDKs having enhanced CDK activity relative to corresponding non-mutated CDKs. The present invention also provides a novel screening method for the identification of non-active CDKs that are able to bind to CKIs (cyclin dependent kinase inhibitors). The invention also provides mutant CDKs obtainable by the screening methods according to the invention.

The ever-increasing world population and the dwindling supply of arable land available for agriculture fuel agricultural research towards improving the efficiency of agriculture. Conventional means for crop and horticultural improvements utilise selective breeding techniques to identify plants having desirable characteristics. However, such selective breeding techniques have several drawbacks, namely that these techniques are typically labour intensive and result in plants that often contain heterogeneous genetic components that may not always result in the desirable trait being passed on from parent plants. Advances in molecular biology have allowed mankind to modify the germplasm of animals and plants. Genetic engineering of plants entails the isolation and manipulation of genetic material (typically in the form of DNA or RNA) and the subsequent introduction of that genetic material into a plant. Such technology has the capacity to deliver crops or plants having various improved economic, agronomic or horticultural traits. A trait of particular economic interest is yield. Yield is normally defined as the measurable produce of economic value from a crop. This may be defined in terms of quantity and/or quality. Yield is directly dependent on several factors, for example, the number and size of the organs, plant architecture (for example, the number of branches), seed production and more. Root development, nutrient uptake and stress tolerance are also important factors in determining yield. Typical stresses to which plants are subjected include environmental (abiotic) stresses (such as temperature stresses caused by atypical high or low temperatures; stresses caused by nutrient deficiency; stresses caused by lack of water (drought)) and biotic stresses (which can be imposed on plants by other plants (weeds), animal pests and pathogens). Crop yield may be increased not only by optimising one of the abovementioned factors, but may also be increased by modifying the inherent growth mechanisms of a plant.

The inherent growth mechanisms of a plant reside in a highly ordered sequence of events collectively known as the ‘cell cycle’. Progression through the cell cycle is fundamental to the growth and development of all multicellular organisms and is crucial to cell proliferation. The major components of the cell cycle are highly conserved in yeast, mammals, and plants. The cell cycle is typically divided into the following sequential phases: G0-G1-S-G2-M. DNA replication or synthesis generally takes place during the S phase (“S” is for DNA synthesis) and mitotic segregation of the chromosomes occurs during the M phase (the “M” is for mitosis), with intervening gap phases, G1 (during which cells grow before DNA replication) and G2 (a period after DNA replication during which the cell prepares for division). Cell division is completed after cytokinesis, the last step of the M phase. Cells that have exited the cell cycle and that have become quiescent are said to be in the G0 phase. Cells in this phase can be stimulated to renter the cell cycle at the G1 phase. The “G” in G1, G2 and G0 stands for “gap”. Completion of the cell cycle process allows each daughter cell during cell division to receive a full copy of the parental genome.

Cell division is controlled by two principal cell cycle events, namely initiation of DNA synthesis and initiation of mitosis. Each transition to each of these key events is controlled by a checkpoint represented by specific protein complexes (involved in DNA replication and division). The expression of genes necessary for DNA synthesis at the G1/S boundary is regulated by the E2F family of transcription factors in mammals and plant cells (La Thangue, 1994 (Curr. Opin. Cell Biol. 6 (3), 443-450); Muller et al., 2001 (Genes Dev. 15 (3), 267-285); De Veylder et al., 2002 (EMBO J. 21 (6), 1360-1368)). Entry into the cell cycle is regulated/triggered by an E2F/Rb complex that integrates signals and allows activation of transcription of cell cycle genes. The transition between the different phases of the cell cycle, and therefore progression through the cell cycle, is driven by the formation and activation of different heterodimeric serine/threonine protein kinases, generally referred to as cyclin-dependent kinases (CDKs). A prerequisite for activity of these kinases is the physical association with a specific cyclin, the timing of activation being largely dependent upon cyclin expression. Cyclin binding induces conformational changes in the N-terminal lobe of the associating CDK and contributes to the localisation and substrate specificity of the complex. Monomeric CDKs are activated when they are associated with cyclins and thus have kinase activity. Cyclin protein levels fluctuate in the cell cycle and therefore represent a major factor in determining timing of CDK activation. The periodic activation of these complexes containing cyclins and CDK during cell cycle mediates the temporal regulation of cell-cycle transitions (checkpoints). Other factors regulating CDK activity include CDK inhibitors (CKIs or ICKs, KIPs, CIPs, INKs), CDK activating kinases (CAKs), a CDK phosphatase (Cdc25) and a CDK subunit (CKS)(Mironov et al. 1999 (Plant Cell 11 (4), 509-522); Reed 1996 (Progressin Cell cycle Research 2, 15-27)).

In plants, two major classes of CDKs, known as A-type and B-type CDKs, have been studied to date. The A-type CDKs regulate both the G1-to-S and G2-to-M transitions, whereas the B-type CDKs seem to control the G2-to-M checkpoint only (Hemerly et al., 1995 (EMBO J. 14 (16), 3925-3936); Magyar et al., 1997 (Plant Cell 9 (2), 223-235); Porceddu et al., 2001 (J. Biol. Chem. 276 (39) 36354-36360)). In addition, the presence of C-type CDKs and CDK-activating kinases (CAKs) has been reported (Magyar et al., 1997 (Plant Cell 9 (2), 223-235); Umeda et al., 1998 (Proc Natl Acad Sci USA. 95 (9), 5021-5026; Joubès et al., 2001 (Plant Physiol. 126 (4), 1403-1415)), as has the presence of D-type, E-type and F-type CDKs (Vandepoele at al. 2002 (Plant Cell 14 (4), 903-916)).

The ability to influence the cell cycle in a plant, and to thereby modify various growth characteristics of a plant, would have many applications in areas such as crop enhancement, plant breeding, production of ornamental plants, aboriculture, horticulture, forestry, the production of algae or plants (for example for use as bioreactors, for the production of substances such as pharmaceuticals, antibodies, or vaccines, or for the bioconversion of organic waste or for use as fuel in the case of high-yielding algae and plants).

It has now been found that modulating expression in a plant of a B-type CDK nucleic acid and/or modulating activity and/or levels in a plant of a B-type CDK protein gives plants having modified growth characteristics. Therefore according to a first embodiment of the present invention there is provided a method for modifying (improving) the growth characteristics of a plant, comprising modulating expression in a plant of a B-type CDK nucleic acid and/or modulating activity and/or levels in a plant of a B-type CDK protein, wherein the modified growth characteristics are selected from increased growth rate, increased yield and modified architecture.

Modulating expression of a B-type CDK nucleic acid is enhancing or increasing expression of a B-type CDK gene/nucleic acid. Modulating activity and/or levels of a B-type CDK protein is increasing activity (which may or may not be as a result of increased levels of a B-type CDK protein). The altered expression, activity and/or levels are altered compared to expression, activity and/or levels of a B-type CDK in corresponding wild-type plants. Methods for obtaining enhanced or increased expression of genes or gene products are well documented in the art and include, for example, overexpression driven by a strong promoter, the use of transcription enhancers or translation enhancers.

A preferred approach for modifying (more specifically improving) the growth characteristics of plants comprises introducing and expressing in a plant a B-type CDK nucleic acid, which nucleic acid encodes a B-type CDK protein. The nucleic acid may be introduced into a plant by, for example, transformation. Therefore, according to a preferred aspect of the present invention, there is provided a method for improving the growth characteristics of a plant comprising introducing into a plant, in an expressible format, a B-type CDK nucleic acid, which B-type CDK nucleic acid encodes a B-type CDK protein, wherein the improved growth characteristics are any one or more of: increased yield, increased growth rate and modified architecture, each relative to corresponding wild-type plants.

A “B-type CDK nucleic acid” as defined herein is a nucleic acid/gene encoding a protein having: (i) a PPTALRE motif with no mismatches or with a mismatch at position 2 and/or 4 from left to right; (ii) a catalytic kinase domain; and (iii) a T-loop activation kinase domain (Magyar et al., 1997 (Plant Cell 9 (2), 223-235)).

The motifs and domains identified in (i) to (iii) above may easily be identified by persons skilled in the art using routine techniques. A kinase assay may, for example, be performed as described in Cockcroft et al., 2000 (Nature, 405 (6786), 575-579). The assay involves grinding plant material in liquid nitrogen and resuspending in an appropriate buffer (such as 1 ml of 50 mM Tris-HCl pH 7.5, 75 mM NaCl, 15 mM EGTA, 15 mMMgCl2, 1 mM dithiothreitol, 0.1% Tween 20, 1×complete Tm protease inhibitor, 1 mMNaF, 0.2 mM NaV, 2 mM Na-pyrophosphate, 60 mM beta-glycerophosphate). The suspension is then homogenized for 4×30 sec with 30 sec on ice between homogeneizations. The supernatant is then incubated with microliters of protein A Sepharose (50% suspension) for 30 min at 4° C. The supernatant is incubated with 1 microliter of antiserum (directed against the carboxy terminal peptide of a CDC2b protein, as described in Setiady et al., 1996 (Plant Cell Physiology 37 (3), 369-376) for 2 h on ice, then 20 microliters protein A-Sepharose added and the sample rotated at 4° C. for an hour. Samples are washed 2 times with kinase buffer (50 mM Tris-HCl pH7.5, 100 mM NaCl, 5 mM EGTA, 1 mM DTT), resuspended in 15 microliters assay buffer (50 mM Tris-HCl pH7.5, 100 mM NaCl, 5 mM EGTA, 10 mM MgCl2, 1 mM DTT, 1 mM NaF, 0.2 mM Na-Vanadate, 2 mM Na pyrophosphate, 25 mM beta glycerophosphate, 0.5 mM histone H1 as a substrate, 0.5 mM PMSF, and 74 kBq [gamma ³²P ATP (>185 TBq mmol-1) per 15 microliters of reaction) and incubated at room temperature for 30 min. The reaction may be stopped by adding gel loading buffer and samples may be analysed using SDS-PAGE and quantified using a phosphoimager (Molecular Dynamics).

The term “B-type CDK amino acid” as defined herein encompasses any amino acid sequence which when used in the construction of a CDK phylogenetic tree, such as the one depicted in FIG. 1, tends to cluster around the B-type CDKs rather than any of the other CDK groups. A person skilled in the art could readily determine whether any amino acid sequence in question falls within the definition of a “B-type CDK amino acid” using known techniques and software for the making of such a phylogenetic tree, such as a GCG, EBI or CLUSTAL package, using default parameters. Upon construction of such a phylogenetic tree, sequences clustering in the B-type CDK group will be considered to fall within the definition of a “B-type CDK” and will therefore be useful in performing the methods of the invention. Additionally or alternatively, a “B-type CDK amino acid” as defined herein is one comprising: (i) a PPTALRE motif with no mismatches or with a mismatch at position 2 and/or 4 from left to right; (ii) a catalytic kinase domain; (iii) a T-loop activation kinase domain (Magyar et al., 1997 (Plant Cell 9 (2), 223-235)).

Table 1 below shows representative B-type CDKs and the PPTALRE motif with no mismatches or with a mismatch at position 2 and/or 4 from left to right.

TABLE 1

| B-type CDK | NCBI Accession Number | Motif |
|---|---|---|
| Arabidopsis CDKB1.1 | At3g54180 | PPTALRE |
| Arabidopsis CDKB1.2 | At2g38620 | PPTALRE |
| Tobacco CDKB1-1 | AF289465 | PPTALRE |
| Tobacco CDKB1-2 | AF289466 | PPTALRE |
| Medicago CDC2MsD | X97315 | PPTALRE |
| Tomato CDKB1 | AJ297916 | PPTALRE |
| Sunflower CDKB1.1 | AY063463 | PPTALRE |
| Antirrhinum CDC2 kinase | X97639 | PPTALRE |
| Chenopodium CDK | AJ278885 | PPTALRE |
| Oryza CDK predicted | NM_190272 | PPTALRE |
| Oryza CDK predicted | D64036 | PPTALRE |
| Maize CDK predicted | AY106440 | PPTALRE |
| Maize CDK predicted | AY106029 | PPTALRE |
| Wheat CDK predicted | BT009182 | PPTALRE |
| Medicago CDC2MSF | X97317 | PPTTLRE |
| Populus CDKB | AY307372 | PPTTLRE |
| Tomato CDKB2 | AJ297917 | PPTTLRE |
| Soybean CDKB | AY439096 | PPTTLRE |
| Antirrhinum CDC2 kinase | X97640 | PPTTLRE |
| Arabidopsis CDKB2.2 | At1g20930 | PPTTLRE |
| Arabidopsia CDKB2.1 | At1g76540 | PSTTLRE |

The B-type CDK nucleic acid/gene may be isolated or derived from any plant or algal or fungal source. This nucleic acid may be substantially modified from its native form in composition and/or genomic environment through deliberate human manipulation. The B-type CDK nucleic acid may be isolated from a monocotyledonous or dicotyledonous species, preferably from the family Brassicaceae, further preferably from Arabidopsis thaliana. The nucleic acid is preferably a class 1 B-type CDK, such as a class 1 B-type CDK selected from the examples of class 1 CDKs shown in FIG. 1, namely, CDK B1;1 from Arabidopsis thaliana, CDK B1;2 from Arabidopsis thaliana, CDKB1;1 from Lycopersicon esculentum (tomato), CDK B1;1 from Antirrhinum majus, CDK B1;1 from Medicago sativa (alfalfa) and CDK B1 from Dunaliella tertiolecta, further preferably the class 1 B-type CDK is a CDK B1;1 from Arabidopsis thaliana or a CDK B1;2 from Arabidopsis thaliana. Alternatively, the nucleic acid is preferably a class 2 B-type CDK, such as a class 2 B-type CDK selected from the examples shown in FIG. 1, namely, a CDK B2;1 from Arabidopsis thaliana, a CDK B2;2 from Arabidopsis thaliana, a CDK B2;1 from Antirrhinum majus, a CDK B2;1 from Mesembryanthemum crassifolium, a CDK B2;1 from Medicago sativa, a CDK B2;1 from Lycopersicon esculentum and a CDK B 1 from Oryza sativa, further preferably the class 2 B-type CDK is a CDK B2;2 from Arabidopsis thaliana.

Most preferably the CDK B1;1 nucleic acid is as represented by SEQ ID NO: 1 or by a portion thereof, or by a nucleic acid sequence capable of hybridising therewith, and wherein the CDK B1;1 protein is as represented by SEQ ID NO: 2, or a homologue, derivative or active fragment thereof. Most preferably the CDK B1;2 nucleic acid is as represented by SEQ ID NO: 3 or by a portion thereof, or by a nucleic acid sequence capable of hybridising therewith, and wherein the CDK B1;2 protein is as represented by SEQ ID NO: 4, or a homologue, derivative or active fragment thereof. Most preferably the CDK B2;2 nucleic acid is as represented by SEQ ID NO: 5 or by a portion thereof, or by a nucleic acid sequence capable of hybridising therewith, and wherein the CDK B2;2 protein is as represented by SEQ ID NO: 6, or a homologue, derivative or active fragment thereof. Each of the CDK B1;1, CDKB1;2 and CDK B2;2 nucleic acids/proteins also encompass the variant nucleic acids and amino acids as described hereinafter.

Although the invention has been exemplified with a B-type CDK according to SEQ ID NO: 1, SEQ ID NO: 3 and SEQ ID NO: 5, and corresponding amino acids according to SEQ ID NO: 2, SEQ ID NO: 4 and SEQ ID NO: 6, respectively, it would be apparent to a person skilled in the art that the methods according to the invention may also be practised using variant nucleic acids and variant amino acids, such as the ones defined hereinafter. Therefore, taken in a broad context, the term “B-type CDK” protein/nucleic acid also encompasses variant nucleic acids and variant amino acids suitable for practising the methods according to the invention. Variant nucleic acids and variant amino acids suitable for practising the methods according to the invention include those falling within the definition of a “B-type CDK”, meaning that upon construction of a phylogenetic tree, such as the one depicted in FIG. 1, the variant sequences of interest would tend to cluster around the B-type CDKs and/or which variant encodes (in the case of a variant nucleic acid) or is (in the case of a variant amino acid) a protein comprising: (i) a PPTALRE motif with no mismatches or with a mismatch at position 2 and/or 4 from left to right; (ii) a catalytic kinase domain; (iii) a T-loop activation kinase domain (Magyar et al., 1997 (Plant Cell 9 (2), 223-235)).

Suitable variant nucleic acid and amino acid sequences useful in practising the method according to the invention, include:
(i) Functional portions of a B-type CDK nucleic acid/gene;
(ii) Sequences capable of hybridising with a B-type CDK nucleic acid/gene;
(iii) Alternative splice variants of a B-type CDK nucleic acid/gene;
(iv) Allelic variants of a B-type CDK nucleic acid/gene;
(v) Homologues, derivatives and active fragments of a B-type CDK protein;
(vi) Mutant B-type CDKs.

An example of a variant B-type nucleic acid/gene is a functional portion of a B-type nucleic acid/gene. It would be apparent to a person skilled in the art that the full length DNA sequence is not a prerequisite to carrying out the methods according to the invention. The methods according to the invention may advantageously be practised using functional portions of a B-type CDK. A functional portion refers to a piece of DNA derived or prepared from an original (larger) B-type CDK DNA molecule, which DNA portion, when introduced and expressed in a plant, gives plants having modified growth characteristics, which portion encodes a protein comprising: (i) a PPTALRE motif with no mismatches or with a mismatch at position 2 and/or 4 from left to right; (ii) a catalytic kinase domain; (iii) a T-loop activation kinase domain (Magyar et al., 1997 (Plant Cell 9 (2), 223-235)). The portion may comprise many genes, with or without additional control elements or may contain spacer sequences. The portion may be made by making one or more deletions and/or truncations to the nucleic acid sequence of, for example, any one of SEQ ID NO: 1, SEQ ID NO: 3 and SEQ ID NO: 5. Techniques for introducing truncations and deletions into a nucleic acid are well known in the art.

An example of a further variant B-type CDK nucleic acid is a sequence that is capable of hybridising to a B-type CDK. Advantageously, the methods according to the present invention may also be practised using sequences capable of hybridising to a B-type CDK, particularly a B-type CDK as represented by any one of SEQ ID NO: 1, SEQ ID NO: 3 and SEQ ID NO: 5, which hybridising sequences are those falling within the definition of a “B-type CDK”, meaning that upon construction of a phylogenetic tree, such as the one depicted in FIG. 1, the hybridising sequence would be one that tends to cluster around the B-type CDKs rather than any of the other CDK groups and/or which hybridising sequence encodes a protein comprising: (i) a PPTALRE motif with no mismatches or with a mismatch at position 2 and/or 4 from left to right; (ii) a catalytic kinase domain; (iii) a T-loop activation kinase domain (Magyar et al., 1997 (Plant Cell 9 (2), 223-235)).

The term “hybridisation” as defined herein is a process wherein substantially homologous complementary nucleotide sequences anneal to each other. The hybridisation process can occur entirely in solution, i.e. both complementary nucleic acids are in solution. Tools in molecular biology relying on such a process include the polymerase chain reaction (PCR; and all methods based thereon), subtractive hybridisation, random primer extension, nuclease S1 mapping, primer extension, reverse transcription, cDNA synthesis, differential display of RNAs, and DNA sequence determination. The hybridisation process can also occur with one of the complementary nucleic acids immobilised to a matrix such as magnetic beads, Sepharose beads or any other resin. Tools in molecular biology relying on such a process include the isolation of poly (A+) mRNA. The hybridisation process can furthermore occur with one of the complementary nucleic acids immobilised to a solid support such as a nitro-cellulose or nylon membrane or immobilised by e.g. photolithography to e.g. a siliceous glass support (the latter known as nucleic acid arrays or microarrays or as nucleic acid chips). Tools in molecular biology relying on such a process include RNA and DNA gel blot analysis, colony hybridisation, plaque hybridisation, in situ hybridisation and microarray hybridisation. In order to allow hybridisation to occur, the nucleic acid molecules are generally thermally or chemically denatured to melt a double strand into two single strands and/or to remove hairpins or other secondary structures from single stranded nucleic acids. The stringency of hybridisation is influenced by conditions such as temperature, salt concentration and hybridisation buffer composition. High stringency conditions for hybridisation include high temperature and/or low salt concentration (salts include NaCl and Na₃-citrate) and/or the inclusion of formamide in the hybridisation buffer and/or lowering the concentration of compounds such as SDS (detergent) in the hybridisation buffer and/or exclusion of compounds such as dextran sulphate or polyethylene glycol (promoting molecular crowding) from the hybridisation buffer. Conventional hybridisation conditions are described in, for example, Sambrook (2001) Molecular Cloning: a laboratory manual, 3rd Edition Cold Spring Harbor Laboratory Press, CSH, New York, but the skilled craftsman will appreciate that numerous different hybridisation conditions can be designed in function of the known or the expected homology and/or length of the nucleic acid sequence. Sufficiently low stringency hybridisation conditions are particularly preferred (at least in the first instance) to isolate nucleic acids heterologous to the DNA sequences of the invention defined supra. An example of low stringency conditions is 4-6×SSC/0.1-0.5% w/v SDS at 37-45° C. for 2-3 hours. Depending on the source and concentration of the nucleic acid involved in the hybridisation, alternative conditions of stringency may be employed, such as medium stringency conditions. Examples of medium stringency conditions include 1-4×SSC/0.25% w/v SDS at ≧45° C. for 2-3 hours. An example of high stringency conditions includes 0.1-1×SSC/0.1% w/v SDS at 60° C. for 1-3 hours. The skilled man will be aware of various parameters which may be altered during hybridisation and washing and which will either maintain or change the stringency conditions. The stringency conditions may start low and be progressively increased until there is provided a hybridising B-type CDK nucleic acid, as defined hereinabove. Elements contributing to heterology include allelism, degeneration of the genetic code and differences in preferred codon usage.

Another example of a variant B-type CDK is an alternative splice variant of a B-type CDK. The methods according to the present invention may also be practised using an alternative splice variant of a B-type CDK nucleic acid/gene. The term “alternative splice variant” as used herein encompasses variants of a nucleic acid in which selected introns and/or exons have been excised, replaced or added. Such splice variants may be found in nature or can be manmade using techniques well known in the art. The splice variants useful in the methods according to the invention are “B-type CDKs”, meaning that upon construction of a phylogenetic tree, such as the one depicted in FIG. 1, the splice variant of interest would be one tending to cluster around the B-type CDKs rather than around any of the other CDK groups and/or which splice variant encodes a protein comprising: (i) a PPTALRE motif with no mismatches or with a mismatch at position 2 and/or 4 from left to right; (ii) a catalytic kinase domain; (iii) a T-loop activation kinase domain (Magyar et al., 1997 (Plant Cell 9 (2), 223-235)). Preferably, the splice variant is a splice variant of the sequence represented by any of SEQ ID NO: 1, SEQ ID NO: 3 and SEQ ID NO: 5

Another example of a variant B-type CDK is an allelic variant. Advantageously, the methods according to the present invention may also be practised using allelic variants of a B-type CDK nucleic acid, preferably an allelic variant of a sequence represented by any of SEQ ID NO: 1, SEQ ID NO: 3 and SEQ ID NO: 5. Allelic variants exist in nature and encompassed within the methods of the present invention is the use of these isolated natural alleles in the methods according to the invention. The allelic variants useful in the methods according to the invention are “B-type CDKs”, meaning that upon construction of a phylogenetic tree, such as the one depicted in FIG. 1, the allelic variant of interest would tend to cluster around the B-type CDKs and/or which allelic variant encodes a protein comprising: (i) a PPTALRE motif with no mismatches or with a mismatch at position 2 and/or 4 from left to right; (ii) a catalytic kinase domain; (iii) a T-loop activation kinase domain (Magyar et al., 1997 (Plant Cell 9 (2), 223-235)).

Examples of variant B-type amino acids include homologues, derivatives and active fragments of a B-type CDK protein. Advantageously, the methods according to the present invention may also be practised using homologues, derivatives or active fragments of a B-type CDK, preferably using nucleic acids encoding homologues, derivatives or active fragments of a B-type CDK as represented by any one of SEQ ID NO: 2, SEQ ID NO: 4 and SEQ ID NO: 6.

“Homologues” of a B-type CDK protein encompass peptides, oligopeptides, polypeptides, proteins and enzymes having amino acid substitutions, deletions and/or insertions relative to the unmodified protein in question and having similar biological and functional activity as the unmodified protein from which they are derived. To produce such homologues, amino acids of the protein may be replaced by other amino acids having similar properties (such as similar hydrophobicity, hydrophilicity, antigenicity, propensity to form or break α-helical structures or β-sheet structures). Conservative substitution tables are well known in the art (see for example Creighton (1984) Proteins. W.H. Freeman and Company). The homologues useful in the methods according to the invention are preferably B-type CDKs, meaning that upon construction of a phylogenetic tree, such as the one depicted in FIG. 1, any homologous sequences of interest would tend to cluster around B-type CDKs rather than any other group of CDKs. Such B-type CDKs have in increasing order of preference at least 60% or 65% or 70% or 75% or 80% or 85% or 90% or 95%, 96%, 97%, 98%, 99% or more, sequence identity or similarity to that of the Arabidopsis thaliana CDK B1;1 (SEQ ID NO: 2) and which homologue comprises: (i) a PPTALRE motif with no mismatches or with a mismatch at position 2 and/or 4 from left to right; (ii) a catalytic kinase domain; (iii) a T-loop activation kinase domain (Magyar et al., 1997 (Plant Cell 9 (2), 223-235)).

Whether a polypeptide has at least 60% identity to the Arabidopsis thaliana CDK B1;1 may readily be established by sequence alignment. Methods for the alignment of sequences for comparison are well known in the art, such methods include GAP, BESTFIT, BLAST, FASTA and TFASTA. GAP uses the algorithm of Needleman and Wunsch 1970 (J. Mol. Biol. 48 (3), 443-453) to find the alignment of two complete sequences that maximises the number of matches and minimises the number of gaps. The BLAST algorithm calculates percent sequence identity and performs a statistical analysis of the similarity between the two sequences. The software for performing BLAST analysis is publicly available through the National Centre for Biotechnology Information. A protein having at least 60% identity to the Arabidopsis thaliana CDK B1;1 may readily be identified by aligning a query sequence with known B-type CDK sequences (see FIG. 1 for example) using, for example, the VNTI AlignX multiple alignment program, based on a modified clustal W algorithm (InforMax, Bethesda, Md., http://www.informaxinc.com), with default settings for gap opening penalty of 10 and a gap extension of 0.05.

Two special forms of homology: orthologs and paralogs, are evolutionary concepts used to describe the ancestral relationships of genes. The term “paralogous” relates to gene-duplications within the genome of a species leading to paralogous genes. The term “orthologous” relates to homologous genes in different organisms due to ancestral relationship. The term “homologues” as used herein also encompasses paralogues and orthologues of the proteins useful in the methods according to the invention.

Othologues in, for example, monocot plant species may easily be found by performing a so-called reciprocal blast search. This may be done by a first blast involving blasting the sequence in question (for example, SEQ ID NO: 1 or SEQ ID NO: 2) against any sequence database, such as the publicly available NCBI database which may be found at: http://www.ncbi.nlm.nih.gov. If orthologues in rice were sought, the sequence in question would be blasted against, for example, the 28,469 full-length cDNA clones from Oryza sativa Nipponbare available at NCBI. BLASTn or tBLASTX may be used when starting from nucleotides or BLASTP or TBLASTN when starting from the protein, with standard default values. The blast results may be filtered. The full-length sequences of either the filtered results or the non-filtered results are then blasted back (second blast) against the sequences of the organism from which the sequence in question is derived. The results of the first and second blasts are then compared. An orthologue is found when the results of the second blast give as hits with the highest similarity a B-type CDK nucleic acid or protein, for example, if one of the organisms is Arabidopsis then a paralogue is found. In the case of large families, ClustalW may be used, followed by a neighbour joining tree, to help visualize the clustering. “Substitutional variants” of a protein are those in which at least one residue in an amino acid sequence has been removed and a different residue inserted in its place. Amino acid substitutions are typically of single residues, but may be clustered depending upon functional constraints placed upon the polypeptide; insertions will usually be of the order of about 1 to 10 amino acid residues and deletions will range from about 1 to 20 residues. Preferably, amino acid substitutions comprise conservative amino acid substitutions. Substitutional variants useful in the methods of the invention will be those comprising: (i) a PPTALRE motif with no mismatches or with a mismatch at position 2 and/or 4 from left to right; (ii) a catalytic kinase domain; and (iii) a T-loop activation kinase domain (Magyar et al., 1997 (Plant Cell 9 (2), 223-235)).

“Insertional variants” of a protein are those in which one or more amino acid residues are introduced into a predetermined site in a protein. Insertions can comprise amino-terminal and/or carboxy-terminal fusions as well as intra-sequence insertions of single or multiple amino acids. Generally, insertions within the amino acid sequence will be smaller than amino- or carboxy-terminal fusions, of the order of about 1 to 10 residues. Examples of amino- or carboxy-terminal fusion proteins or peptides include the binding domain or activation domain of a transcriptional activator as used in the yeast two-hybrid system, phage coat proteins, (histidine)₆-tag, glutathione S-transferase-tag, protein A, maltose-binding protein, dihydrofolate reductase, Tag•100 epitope, c-myc epitope, FLAG®-epitope, lacZ, CMP (calmodulin-binding peptide), HA epitope, protein C epitope and VSV epitope. Insertional variants useful in the methods of the invention will be those comprising: (i) a PPTALRE motif with no mismatches or with a mismatch at position 2 and/or 4 from left to right; (iii) a T-loop activation kinase domain (Magyar et al., 1997 (Plant Cell 9 (2), 223-235)).

“Deletion variants” of a protein are characterised by the removal of one or more amino acids from the protein. Amino acid variants of a protein may readily be made using peptide synthetic techniques well known in the art, such as solid phase peptide synthesis and the like, or by recombinant DNA manipulations. Methods for the manipulation of DNA sequences to produce substitution, insertion or deletion variants of a protein are well known in the art. For example, techniques for making substitution mutations at predetermined sites in DNA are well known to those skilled in the art and include M13 mutagenesis, T7-Gen in vitro mutagenesis (USB, Cleveland, Ohio), QuickChange Site Directed mutagenesis (Stratagene, San Diego, Calif.), PCR-mediated site-directed mutagenesis or other site-directed mutagenesis protocols. Deletional variants useful in the methods of the invention will be those comprising: (i) a PPTALRE motif with no mismatches or with a mismatch at position 2 and/or 4 from left to right; (ii) a catalytic kinase domain; (iii) a T-loop activation kinase domain (Magyar et al., 1997 (Plant Cell 9 (2), 223-235)).

Methods for the search and identification of B-type CDK homologues would be well within the realm of a person skilled in the art. Methods for the alignment of sequences for comparison are well known in the art. Such methods include GAP, BESTFIT, BLAST, FASTA and TFASTA. GAP uses the algorithm of Needleman and Wunsch 1970 (J. Mol. Biol. 48 (3), 443-453) to find the alignment of two complete sequences that maximises the number of matches and minimises the number of gaps. The BLAST algorithm calculates percent sequence identity and performs a statistical analysis of the similarity between the two sequences. The software for performing BLAST analysis is publicly available through the National Centre for Biotechnology Information.

The term “derivatives” refers to peptides, oligopeptides, polypeptides, proteins and enzymes which may comprise substitutions, deletions or additions of naturally and non-naturally occurring amino acid residues compared to the amino acid sequence of a naturally-occurring form of the protein, for example, as represented by any one of SEQ ID NO: 2, SEQ ID NO: 4 and SEQ ID NO: 6. “Derivatives” of a B-type CDK protein encompass peptides, oligopeptides, polypeptides, proteins and enzymes which may comprise naturally occurring altered, glycosylated, acylated or non-naturally occurring amino acid residues compared to the amino acid sequence of a naturally-occurring form of the polypeptide. A derivative may also comprise one or more non-amino acid substitutents compared to the amino acid sequence from which it is derived, for example a reporter molecule or other ligand, covalently or non-covalently bound to the amino acid sequence such as, for example, a reporter molecule which is bound to facilitate its detection, and non-naturally occurring amino acid residues relative to the amino acid sequence of a naturally-occurring protein. Derivatives useful in the methods of the invention will be those comprising: (i) a PPTALRE motif with no mismatches or with a mismatch at position 2 and/or 4 from left to right; (ii) a catalytic kinase domain; (iii) a T-loop activation kinase domain (Magyar et al., 1997 (Plant Cell 9 (2), 223-235)).

“Active fragments” of a B-type CDK protein comprise: (i) a PPTALRE motif with no mismatches or with a mismatch at position 2 and/or 4 from left to right; (ii) a catalytic kinase domain; (iii) a T-loop activation kinase domain (Magyar of al., 1997 (Plant Cell 9 (2), 223-235)).

Further advantageously, the methods according to the present invention may also be practised using mutant plant CDKs, which mutant CDKs have at least a substantially similar, preferably enhanced, biological activity compared with corresponding wild-type CDK proteins.

The present invention further provides a hitherto unknown screening method for the identification of plant CDKs having enhanced CDK activity relative to corresponding non-mutated or wild-type CDKs. These plant-derived CDK mutants having enhanced CDK activity may be used in the methods according to the invention or may find uses in other areas.

Therefore according to a second embodiment of the present invention, there is provided a screening method for the identification of mutant plant CDKs having substantially similar or enhanced CDK activity relative to corresponding non-mutated CDKs, which method comprises the steps of:
(i) Providing plant-derived CDK mutants;
(ii) Identifying ICK non-reacting mutants;
(iii) Identifying mutants having cyclin-binding activity; and optionally followed by,
(iv) A yeast complementation assay on resultant mutants from steps (ii) and (ii).

The substantially similar or enhanced CDK activity is in the presence of plant ICKs.

The novel screening method may be preceded with steps for mutating wild-type CDKs, if necessary. These steps comprise:
(a) Providing wild-type CDK amino acids; and
(b) Mutating substantially each CDK amino acid at least at one amino acid position.

A mutation may be introduced into the CDK using conventional techniques, such as error prone PCR. The mutation may be introduced randomly or by site directed mutagenesis.

Although the method for the identification of CDK mutants has been exemplified with mutant CDK A-type proteins, the method is equally well suited to the identification of other mutant CDKs.

Examples of particularly preferred mutants (mutants 1 to 3) obtainable by the above screening method according to the invention are listed in Table A below. The mutants are mutants of an A-type CDK, namely an A;1 CDK from rice (see SEQ ID NO: 8). These mutants may be of particular use in the methods for modifying the growth characteristics of plants using the novel methods as described hereinabove.

Alternatively, a CDK having a mutation consisting of at least one of the seven amino acid changes identified in Table A may also be useful in the methods according to the invention. The suitability of such a mutant in the methods according to the invention may readily be determined by passing the mutants through the novel screening method identified above so as to determine whether the mutant has substantially similar or enhanced CDK activity relative to corresponding non-mutated CDKs.

TABLE A
Mutants that bind to cyclin, but not to ICKs

| | | |
|---|---|---|
| Mutant | SEQ ID NO | Mutation Position |

| | | | | |
|---|---|---|---|---|
| 1 | SEQ ID NO: 9 | Y4H | V79D | A152T |
| 2 | SEQ ID NO: 10 | I30T | | |
| 3 | SEQ ID NO: 11 | E5V | R122S | K143E |

The mutants are denoted by a change in the appropriate amino acid residue. In the case of mutant 1, for example, at position 4, the Y is substituted for an H; at position 79, the V is substituted for a D; and at position 152 the A is substituted for a T. Mutation positions are calculated from the first methionine of SEQ ID NO: 8.

Therefore according to another embodiment of the present invention, there is provided a method for modifying the growth characteristics of plants, comprising modulating, preferably increasing, activity and/or levels of a CDK mutant comprising at least one of the seven amino acid changes identified in Table A above. The mutated amino acid itself may be introduced directly into a plant cell or into the plant itself (including introduction into a tissue, organ or any other part of a plant).

Also of interest are CDKs with no activity but that are still able to bind to CKI, therefore constituting CKI traps. This hitherto unknown method comprises the steps of:
(i) Providing CDK mutants;
(ii) Identifying ICK binding mutants; and
(iii) Identifying non-cyclin-binding mutants.

Each of steps (i) to (iii) is carried out in the aforementioned method.

The novel screening method may be preceded with steps for mutating wild-type CDKs, if necessary. These steps comprise:
(a) Providing wild-type CDK amino acids; and
(b) Mutating substantially each CDK amino acid at least at one amino acid position.

A mutation may be introduced into the CDK using conventional techniques, such as error prone PCR. The mutation may be introduced randomly or by site directed mutagenesis. Particularly suitable mutant CDKs obtainable by the abovementioned screening method are listed in Table B below. The mutants are mutants of an A-type CDK, namely an A;1 CDK from Arabidopsis thaliana (see SEQ ID NO: 7 and SEQ ID NO: 8).

TABLE B
Mutants that bind CKI but not cyclin

| Mutant | SEQ ID NO | Mutant Position | | | | | | |
|---|---|---|---|---|---|---|---|---|
| 4 | SEQ ID NO: 12 | G154R | | | | | | |
| 5 | SEQ ID NO: 13 | Q3P | E38A | R137L | S182A | I193T | M267V | R279Q |

Mutations positions are calculated from the first methionine of SEQ ID NO: 8.

According to a third embodiment of the present invention, there is provided an isolated CDK nucleic acid molecule comprising:
(a) a nucleic acid encoding a CDK mutant represented by any one of SEQ ID NO: 9, SEQ ID NO: 10, SEQ ID NO: 11, SEQ ID NO: 12 and SEQ ID NO: 13;
(b) a nucleic acid encoding a homologue, derivative or active fragment of a CDK mutant represented by any one of SEQ ID NO: 9, SEQ ID NO: 10, SEQ ID NO: 11, SEQ ID NO: 12 and SEQ ID NO: 13, which homologue, derivative or active fragment comprises at least one of the seven amino acid position changes shown in Table A or at least one of the eight amino acid position changes shown in Table B;
(c) a nucleic acid capable of hybridising with a nucleic acid of (a) or (b) above, wherein the hybridising sequence encodes a protein comprising at least one of the seven amino acid position changes shown in Table A or at least one of the eight amino acid position changes shown in Table B;
(d) a nucleic acid of (a) to (c) above which is degenerate as a result of the genetic code;
(e) allelic variants of the nucleic acids of (a) to (d), which allelic variant encodes a protein comprising at least one of the seven amino acid position changes shown in Table A or at least one of the eight amino acid position changes shown in Table B; and
(f) alternative splice variants of the nucleic acids of (a) to (e), which alternative splice variants encode a protein comprising at least one of the seven amino acid position changes shown in Table A or at least one of the eight amino acid position changes shown in Table B.

According to a fourth embodiment of the present invention, there is provided a CDK mutant, comprising:
(a) an amino acid represented by any one of SEQ ID NO: 9, SEQ ID NO: 10, SEQ ID NO: 11, SEQ ID NO: 12 and SEQ ID NO: 13; and
(b) a fragment of an amino acid of SEQ ID NO: 9, SEQ ID NO: 10, SEQ ID NO: 11, SEQ ID NO: 12 and SEQ ID NO: 13, which fragment comprises at least one of the seven amino acid position changes shown in Table A or at least one of the eight amino acid position changes shown in Table B.

According to a fifth embodiment of the present invention, genetic constructs and vectors to facilitate introduction and/or expression of the nucleotide sequences useful in the methods according to the invention are provided. Therefore, according to a fifth embodiment of the present invention, there is provided a gene construct comprising:
(i) a B-type CDK gene/nucleic acid; or
(ii) a nucleic acid encoding a CDK mutant, which CDK mutant comprises at least one of the seven amino acid position changes shown in Table A or at least one of the eight amino acid position changes shown in Table B;
(iii) one or more control sequences capable of driving expression of the nucleic acid of (i) or (ii); and optionally
(iv) a transcription termination sequence.

Constructs useful in the methods according to the present invention may be constructed using recombinant DNA technology well known to persons skilled in the art. The gene constructs may be inserted into vectors, which may be commercially available, suitable for transforming into plants and suitable for expression of the gene of interest in the transformed cells.

The nucleic acid encoding a CDK mutant may be any of the mutant-encoding nucleic acids described hereinbefore.

The B-type CDK gene/nucleic acid may be isolated from a monocotyledonous or dicotyledonous species, preferably from the family Brassicaceae, further preferably from Arabidopsis thaliana. The nucleic acid is preferably a class 1 B-type CDK, such as a class 1 B-type CDK selected from the examples of class 1 CDKs shown in FIG. 1, namely, CDK B1;1 from Arabidopsis thaliana, CDK B1;2 from Arabidopsis thaliana, CDKB1;1 from Lycopersicon esculentum (tomato), CDK B1;1 from Antirrhinum majus, CDK B1;1 from Medicago sativa (alfalfa) and CDK B1 from Dunaliella tertiolecta. Further preferably the class 1 B-type CDK is a CDK B1;1 from Arabidopsis thaliana or a CDK B1;2 from Arabidopsis thaliana. Alternatively, the nucleic acid is preferably a class 2 B-type CDK, such as a class 2 B-type CDK selected from the examples shown in FIG. 1, namely, a CDK B2;1 from Arabidopsis thaliana, a CDK B2;2 from Arabidopsis thaliana, a CDK B2;1 from Antirrhinum majus, a CDK B2;1 from Mesembryanthemum crassifolium, a CDK B2;1 from Medicago sativa, a CDK B2;1 from Lycopersicon esculentum and a CDK B 1 from Oryza sativa. Further preferably the class 2 B-type CDK is a CDK B2;2 from Arabidopsis thaliana.

Most preferably the CDK B1;1 nucleic acid is as represented by SEQ ID NO: 1 or by a portion thereof, or by a nucleic acid sequence capable of hybridising therewith, and wherein the CDK B1;1 protein is as represented by SEQ ID NO: 2, or a homologue, derivative or active fragment thereof. Most preferably the CDK B1;2 nucleic acid is as represented by SEQ ID NO: 3 or by a portion thereof, or by a nucleic acid sequence capable of hybridising therewith, and wherein the CDK B1;2 protein is as represented by SEQ ID NO: 4, or a homologue, derivative or active fragment thereof. Most preferably the CDK B2;2 nucleic acid is as represented by SEQ ID NO: 5 or by a portion thereof, or by a nucleic acid sequence capable of hybridising therewith, and wherein the CDK B2;2 protein is as represented by SEQ ID NO: 6, or a homologue, derivative or active fragment thereof. Each of the CDK B1;1, CDKB1;2 and CDK B2;2 nucleic acids/proteins also encompass the variant nucleic acids and amino acids as described hereinbefore.

Plants are then transformed with a construct or vector comprising the sequence of interest (i.e., a B-type CDK nucleic acid encoding a B-type CDK protein or a nucleic acid encoding a CDK mutant), which sequence is operably linked to one or more control sequences (at least a promoter).

The terms “regulatory element”, “control sequence” and “promoter” are all used interchangeably herein and are taken to refer to regulatory nucleic acid sequences capable of effecting expression of the sequences to which they are ligated. Encompassed by the aforementioned terms are transcriptional regulatory sequences derived from a classical eukaryotic genomic gene (including the TATA box which is required for accurate transcription initiation, with or without a CCAAT box sequence) and additional regulatory elements (i.e. upstream activating sequences, enhancers and silencers) which alter gene expression in response to developmental and/or external stimuli, or in a tissue-specific manner. Also included within the term is a transcriptional regulatory sequence of a classical prokaryotic gene, in which case it may include a—3 box sequence and/or—10 box transcriptional regulatory sequences. The term “regulatory element” also encompasses a synthetic fusion molecule or derivative which confers, activates or enhances expression of a nucleic acid molecule in a cell, tissue or organ. The terms “control sequence”, “regulatory sequence”, “regulatory element” and “promoter” are used interchangeably herein. The term “operably linked” as used herein refers to a functional linkage between the promoter sequence and the gene of interest, such that the promoter sequence is able to initiate transcription of the gene of interest.

Advantageously, the B-type CDK nucleic acid may be operably linked to any promoter. Preferably, in the case of a CDK B1;1, expression is driven by a promoter active in young, expanding tissue, such as young leaves, flowers, stems and roots. Such a “Young expanding tissue-preferred promoter” as defined herein refers to a promoter that is expressed predominantly in young expanding tissue, but not necessarily exclusively in such tissue. Preferably, the “young expanding tissue-preferred promoter” is the beta-expansin EXPB8 promoter from rice. Other suitable promoters include any expansin promoter, pLEAFY and others. Preferably in the case of a CDK B1;2 and CDK B2;2 expression is driven in a constitutive manner, most preferably wherein the constitutive promoter is a GOS2 promoter. A constitutive promoter is transcriptionally active during most, but not necessarily all, phases of its growth and development. Examples of constitutive plant promoters are given in Table C as shown below. The promoters shown in Table C below may advantageously be used to practise the methods according to the invention.

TABLE C
Examples of Constitutive Promoters

| Gene Source | Expression Pattern | Reference |
|---|---|---|
| Actin | Constitutive | McElroy et al., Plant Cell, 2: |
| | | 163-171, 1990 |
| CAMV 35S | Constitutive | Odell et al., Nature, 313: |
| | | 810-812, 1985 |
| CaMV 19S | Constitutive | Nilsson et al., Physiol. Plant. |
| | | 100: 456-462, 1997 |
| GOS2 | Constitutive | de Pater et al., Plant J Nov; 2(6): |
| | | 837-44, 1992 |
| Ubiquitin | Constitutive | Christensen et al., Plant Mol. |
| | | Biol. 18: 675-689, 1992 |
| Rice cyclophilin | Constitutive | Buchholz et al., Plant Mol Biol. |
| | | 25(5): 837-43, 1994 |
| Maize H3 histone | Constitutive | Lepetit et al., Mol. Gen. Genet. |
| | | 231: 276-285, 1992 |
| Actin 2 | Constitutive | An et al., Plant J. 10(1); |
| | | 107-121, 1996 |

Inducible promoters are promoters that have induced or increased transcription initiation in response to a developmental, chemical, environmental or physical stimulus. For example stress-inducible promoters are activated when a plant is exposed to various stress conditions. Examples of stress-inducible promoters, which are also suitable to practise the methods according to the invention, are given in Table D as shown below. Such promoters may also be useful in practising the methods of the invention since modified growth (such as increased growth) induced in times of stress may have many advantages.

TABLE D
Examples of Stress-Inducible Promoters

| Name | Stress | Reference |
|---|---|---|
| P5CS (delta(1)-pyrroline- | Salt, water | Zhang et al.; Plant Science. |
| 5-carboxylate syntase) | | Oct. 28 1997; 129(1): 81-89 |
| cor15a | Cold | Hajela et al., Plant Physiol. |
| | | 93: 1246-1252 (1990) |
| cor15b | Cold | Wlihelm et al., Plant Mol |
| | | Biol. 1993 December; |
| | | 23(5): 1073-7 |
| cor15a (−305 to +78 nt) | Cold, drought | Baker et al., Plant Mol |
| | | Biol. 1994 March; 24(5): |
| | | 701-13 |
| rd29 | Salt, drought, | Kasuga et al., Nature |
| | cold | Biotechnology, vol 18, |
| | | 287-291, 1999 |
| Heat shock proteins, | Heat | Barros et al., Plant Mol |
| including artificial | | Biol, 19(4): 665-75, 1992. |
| promoters containing the | | Marrs et al., Dev Genet., |
| heat shock element (HSE) | | 14(1): 27-41, 1993. Schoffl |
| | | et al., Mol Gen Gent, |
| | | 217(2-3): 246-53, 1989. |
| smHSP (small heat shock | Heat | Waters et al., J |
| proteins) | | Experimental Botany, vol |
| | | 47, 296, 325-338, 1996 |
| wcs120 | Cold | Quellet et al., FEBS Lett. |
| | | 423, 324-328 (1998) |
| ci7 | Cold | Kirch et al., Plant Mol Biol, |
| | | 33(5): 897-909, 1997 |
| | | March |
| Adh | Cold, drought, | Dolferus et al., Plant |
| | hypoxia | Physiol, 105(4): 1075-87, |
| | | 1994 August |
| pwsi18 | Water: salt | Joshee et al., Plant Cell |
| | and drought | Physiol, 39(1): 64-72, |
| | | 1998, January |
| ci21A | Cold | Schneider et al., Plant |
| | | Physiol, 113(2): 335-45, |
| | | 1997 |
| Trg-31 | Drought | Chaudhary et al., Plant Mol |
| | | Biol, 30(6): 1247-57, 1996 |
| Osmotin | Osmotic | Raghothama et al., Plant |
| | | Mol Biol, 23(6): 1117-28, |
| | | 1993 |
| LapA | Wounding, | WO99/03977 University of |
| | environmental | California/NRA |

The promoters listed in Tables C and D are provided for the purposes of exemplification only and the present invention is not to be limited by the list provided therein. Those skilled in the art will readily be in a position to provide additional promoters that are useful in performing the present invention. The promoters listed may also be modified to provide specificity of expression as required.

Optionally, one or more terminator sequences may also be used in the construct introduced into a plant. The term “terminator” encompasses a control sequence which is a DNA sequence at the end of a transcriptional unit which signals 3′ processing and polyadenylation of a primary transcript and termination of transcription. Additional regulatory elements may include transcriptional as well as translational enhancers. Those skilled in the art will be aware of terminator and enhancer sequences which may be suitable for use in performing the invention. Such sequences would be known or may readily be obtained by a person skilled in the art.

The genetic constructs of the invention may further include an origin of replication sequence which is required for maintenance and/or replication in a specific cell type. One example is when a genetic construct is required to be maintained in a bacterial cell as an episomal genetic element (e.g. plasmid or cosmid molecule). Preferred origins of replication include, but are not limited to, the f1-ori and colE1.

The genetic construct may optionally comprise a selectable marker gene. As used herein, the term “selectable marker gene” includes any gene which confers a phenotype on a cell in which it is expressed to facilitate the identification and/or selection of cells which are transfected or transformed with a nucleic acid construct of the invention. Suitable markers may be selected from markers that confer antibiotic or herbicide resistance. Cells containing the recombinant DNA will thus be able to survive in the presence of antibiotic or herbicide concentrations that kill untransformed cells. Examples of selectable marker genes include the bar gene which provides resistance to the herbicide Basta; the npt gene which confers resistance to the antibiotic kanamycin; the hpt gene which confers hygromycin resistance. Visual markers, such as the Green Fluorescent Protein (GFP, Haseloff et al., 1997 (Proc Natl Acad Sci USA. 94 (6), 2122-2127.), β-glucuronidase (GUS) or luciferase may also be used as selectable markers. Further examples of suitable selectable marker genes include the ampicillin resistance (Ampr), tetracycline resistance gene (Tcr), bacterial kanamycin resistance gene Kanr), phosphinothricin resistance gene, neomycin phosphotransferase gene (nptll), hygromycin resistance gene, gene, and the chloramphenicol acetyltransferase (CAT) gene, amongst others.

The present invention also encompasses plants obtainable by the methods according to the present invention. The present invention therefore provides plants obtainable by the methods according to the present invention, which plants have improved growth characteristics selected from one or more of increased yield, increased growth rate and modified architecture and which plants have increased expression and/or activity and/or levels of a B-type CDK.

The present invention also provides plants having improved growth characteristics selected from one or more of increased yield, increased growth rate and modified architecture, which plants have increased expression and/or levels and/or activity of a B-type CDK.

According to a sixth embodiment of the present invention, there is provided a method for the production of transgenic plants having improved growth characteristics selected from one or more of increased yield, increased growth rate and modified architecture, comprising introduction and expression in a plant of a nucleic acid molecule of the invention.

More specifically, the present invention provides a method for the production of transgenic plants having modified growth characteristics, which method comprises:
(i) introducing and expressing in a plant or a plant cell a B-type CDK gene/nucleic acid; or
(ii) a nucleic acid encoding a CDK mutant, which CDK mutant comprises at least one of the seven amino acid position changes shown in Table A;
(iii) cultivating the plant cell under conditions promoting regeneration and mature plant growth.

The nucleic acid itself may be introduced directly into a plant cell or into the plant itself (including introduction into a tissue, organ or any other part of the plant). According to a preferred feature of the present invention, the nucleic acid is preferably introduced into a plant by transformation.

The nucleic acid encoding a CDK mutant may be any of the mutant-encoding nucleic acids described hereinbefore.

The B-type CDK nucleic acid/gene is preferably a class 1 B-type CDK, such as a class 1 B-type CDK selected from the examples of class 1 CDKs shown in FIG. 1, namely, CDK B1;1 from Arabidopsis thaliana, CDK B1;2 from Arabidopsis thaliana, CDKB1;1 from Lycopersicon esculentum (tomato), CDK B1;1 from Antirrhinum majus, CDK B1;1 from Medicago sativa (alfalfa) and CDK B1 from Dunaliella tertiolecta. Further preferably the class 1 B-type CDK is a CDK B1;1 from Arabidopsis thaliana or a CDK B1;2 from Arabidopsis thaliana. Alternatively and preferably, the nucleic acid is a class 2 B-type CDK, such as a class 2 B-type CDK selected from the examples shown in FIG. 1, namely, a CDK B2;1 from Arabidopsis thaliana, a CDK B2;2 from Arabidopsis thaliana, a CDK B2;1 from Antirrhinum majus, a CDK B2;1 from Mesembryanthemum crassifolium, a CDK B2;1 from Medicago sativa, a CDK B2;1 from Lycopersicon esculentum and a CDK B 1 from Oryza sativa. Further preferably the class 2 B-type CDK is a CDK B2;2 from Arabidopsis thaliana.

Most preferably the CDK B1;1 nucleic acid is as represented by SEQ ID NO: 1 or by a portion thereof, or by a nucleic acid sequence capable of hybridising therewith, and wherein the CDK B1;1 protein is as represented by SEQ ID NO: 2, or a homologue, derivative or active fragment thereof. Most preferably the CDK B1;2 nucleic acid is as represented by SEQ ID NO: 3 or by a portion thereof, or by a nucleic acid sequence capable of hybridising therewith, and wherein the CDK B1;2 protein is as represented by SEQ ID NO: 4, or a homologue, derivative or active fragment thereof. Most preferably the CDK B2;2 nucleic acid is as represented by SEQ ID NO: 5 or by a portion thereof, or by a nucleic acid sequence capable of hybridising therewith, and wherein the CDK B2;2 protein is as represented by SEQ ID NO: 6, or a homologue, derivative or active fragment thereof. Each of the CDK B1;1, CDKB1;2 and CDK B2;2 nucleic acids/proteins also encompass the variant nucleic acids and amino acids as described hereinbefore.

The term “transformation” as referred to herein encompasses the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated therefrom. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid. Alternatively, it may be integrated into the host genome. The resulting transformed plant cell can then be used to regenerate a transformed plant in a manner known to persons skilled in the art.

Transformation of a plant species is now a fairly routine technique. Advantageously, any of several transformation methods may be used to introduce the gene of interest into a suitable ancestor cell. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts (Krens, F. A. et al., 1982, Nature 296, 72-74; Negrutiu I. et al., June 1987, Plant Mol. Biol. 8, 363-373); electroporation of protoplasts (Shillito R. D. et al., 1985 Bio/Technol 3, 1099-1102); microinjection into plant material (Crossway A. et al., 1986, Mol. Gen Genet 202, 179-185); DNA or RNA-coated particle bombardment (Klein T. M. et al., 1987, Nature 327, 70) infection with (non-integrative) viruses and the like. Transgenic rice plants expressing a B-type CDK gene are preferably produced via Agrobacterium-mediated transformation using any of the well known methods for rice transformation, such as described in any of the following: published European patent application EP 1198985 A1, Aldemita and Hodges (Planta, 199, 612-617, 1996); Chan et al. (Plant Mol. Biol. 22 (3) 491-506, 1993), Hiei et al. (Plant J. 6 (2) 271-282, 1994), which disclosures are incorporated by reference herein as if fully set forth. In the case of corn transformation, the preferred method is as described in either Ishida et al. (Nat. Biotechnol. 1996 June; 14(6): 745-50) or Frame et al. (Plant Physiol. 2002 May; 129(1): 13-22), which disclosures are incorporated by reference herein as if fully set forth.

Generally after transformation, plant cells or cell groupings are selected for the presence of one or more markers which are encoded by plant-expressible genes co-transferred with the gene of interest, following which the transformed material is regenerated into a whole plant.

Following DNA transfer and regeneration, putatively transformed plants may be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed to give homozygous second generation (or T2) transformants, and the T2 plants further propagated through classical breeding techniques.

The generated transformed organisms may take a variety of forms. For example, they may be chimeras of transformed cells and non-transformed cells; conal transformants (e.g., all cells transformed to contain the expression cassette); grafts of transformed and untransformed tissues (e.g., in plants, a transformed rootstock grafted to an untransformed scion).

The present invention clearly extends to any plant cell or plant produced by any of the methods described herein, and to all plant parts and propagules thereof. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced in the parent by the methods according to the invention. The invention also includes host cells containing an isolated nucleic acid molecule encoding a protein capable of modulating a B-type CDK protein, preferably wherein the protein is a B-type CDK protein. Preferred host cells according to the invention are plant cells. The invention also extends to harvestable parts of a plant, such as but not limited to, seeds, leaves, fruits, flowers, stem cultures, rhizomes, tubers and bulbs.

The term “plant” as used herein encompasses whole plants, ancestors and progeny of the plants and plant parts, including seeds, shoots, stems, roots (including tubers), and plant cells, tissues and organs. The term “plant” also therefore encompasses suspension cultures, embryos, meristematic regions, callus tissue, leaves, gametophytes, sporophytes, pollen, and microspores. Plants that are particularly useful in the methods of the invention include algae, ferns, and all plants which belong to the superfamily Viridiplantae, in particular monocotyledonous and dicotyledonous plants, including a fodder or forage legumes, ornamental plants, food crops, trees, or shrubs selected from the list comprising Abelmoschus spp., Acer spp., Actinidia spp., Agropyron spp., Allium spp., Amaranthus spp., Ananas comosus, Annona spp., Apium graveolens, Arabidopsis thaliana, Arachis spp, Artocarpus spp., Asparagus officinalis, Avena sativa, Averrhoa carambola, Benincasa hispida, Bertholletia excelsea, Beta vulgaris, Brassica spp., Cadaba farinosa, Camellia sinensis, Canna indica, Capsicum spp., Carica papaya, Carissa macrocarpa, Carthamus tinctorius, Carya spp., Castanea spp., Cichorium endivia, Cinnamomum spp., Citrullus lanatus, Citrus spp., Cocos spp., Coffea spp., Cola spp., Colocasia esculenta, Corylus spp., Crataegus spp., Cucumis spp., Cucurbita spp., Cynara spp., Daucus carota, Desmodium spp., Dimocarpus longan, Dioscorea spp., Diospyros spp., Echinochloa spp., Eleusine coracana, Eriobotrya japonica, Eugenia uniflora, Fagopyrum spp., Fagus spp., Ficus carica, Fortunella spp., Fragaria spp., Ginkgo biloba, Glycine spp., Gossypium hirsutum, Helianthus spp., Hibiscus spp., Hordeum spp., Ipomoea batatas, Juglans spp., Lactuca sativa, Lathyrus spp., Lemna spp., Lens culinaris, Linum usitatissimum, Litchi chinensis, Lotus spp., Luffa acutangula, Lupinus spp., Macrotyloma spp., Malpighia emarginata, Malus spp., Mammea americana, Mangifera indica, Manihot spp., Manilkara zapota, Medicago sativa, Melilotus spp., Mentha spp., Momordica spp., Morus nigra, Musa spp., Nicotiana spp., Olea spp., Opuntia spp., Ornithopus spp., Oryza spp., Panicum miliaceum, Passiflora edulis, Pastinaca sativa, Persea spp., Petroselinum crispum, Phaseolus spp., Phoenix spp., Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Rubus spp., Saccharum spp., Sambucus spp., Secale cereale, Sesamum spp., Solanum spp., Sorghum bicolor, Spinacia spp., Syzygium spp., Tamarindus indica, Theobroma cacao, Trifolium spp., Tritifcosecale rimpaui, Triticum spp., Vaccinium spp., Vicia spp., Vigna spp., Vitis spp., Zea mays, Zizania palustis, Ziziphus spp., amongst others.

According to a preferred feature of the present invention, the plant is a crop plant, such as soybean, sunflower, canola, alfalfa, rapeseed or cotton. Further preferably, the plant according to the present invention is a monocotyledonous plant, such as sugarcane. Most preferably, the plant is a cereal, such as rice, maize, wheat, sorghum, millet or barley.

Advantageously, performance of the methods according to the present invention results in plants having a variety of modified growth characteristics, such modified growth characteristics including, increased yield, increased growth rate and modified architecture, each relative to corresponding wild type plants.

The term “increased yield” as defined herein encompasses an increase in biomass (weight) in one or more parts of a plant, particularly aboveground (harvestable) parts, increased root biomass or increased biomass of any other harvestable part, relative to the biomass of the corresponding parts of corresponding wild-type plants. The term also encompasses an increase in seed yield, which includes an increase in the biomass of the seed (seed weight) and which may be an increase in the seed weight per plant or on an individual seed basis, and/or an increase in the number of (filled) seeds and/or in the size of the seeds and/or an increase in seed volume, each relative to corresponding wild-type plants. An increase in seed size and/or volume may also influence the composition of seeds. An increase in seed yield could be due to an increase in the number and/or size of flowers. An increase in yield might also increase the harvest index, which is expressed as a ratio of the total biomass over the yield of harvestable parts, such as seeds. An increase in yield may also increase the thousand kernel weight (TKW) which is extrapolated from the total weight of the number of filled seeds. An increase in TKW may result from an increased seed size and/or seed weight.

Taking corn as an example, a yield increase may be manifested as one or more of the following: increase in the number of plants per hectare or acre, an increase in the number of ears per plant, an increase in the number of rows, number of kernels per row, kernel weight, thousand kernel weight, ear length/diameter, among others. Taking rice as an example, a yield increase may be manifested by an increase in one or more of the following: number of plants per hectare or acre, number of panicles per plant, number of spikelets per panicle, number of flowers per panicle, increase in the seed filling rate, increase in thousand kernel weight, among others. An increase in yield may also result in modified architecture, or may occur as a result of modified architecture.

According to a preferred feature of the present invention, performance of the methods of the invention result in plants having modified yield. Preferably, the modified yield includes at least one of: an increase in area, an increase in the number of panicles, an increase in height, an increase in the number of seeds, an increase in the number of filled seeds, an increase in the total weight of seeds, an increase in thousand kernel weight (TKW) and an increase in harvest index, each relative to control plants. Therefore, according to the present invention, there is provided a method for increasing yield of plants, which method comprises modulating expression in a plant of a B-type CDK and/or modulating activity and/or levels in a plant of a B-type CDK protein.

Since the transgenic plants according to the present invention have increased yield, it is apparent that these plants exhibit an increased growth rate (during at least part of their life cycle), relative to the growth rate of corresponding wild type plants. The increased growth rate may be specific to one or more parts of a plant (including seeds), or throughout the whole plant. A plant having increased growth rate may even exhibit early flowering. The increase in growth rate may take place at one or more stages in the life cycle of a plant or during substantially the whole plant life cycle. Increased growth rate during the early stages in the life cycle of a plant may reflect enhanced vigour. The increase in growth rate may alter the harvest time of a plant allowing plants to be harvested sooner than would otherwise be possible. If the growth rate is sufficiently increased, it may allow for the sowing of further seeds of the same plant species (for example sowing and harvesting of rice plants followed by sowing and harvesting of further rice plants all within one conventional growing period) or of different plants species (for example the sowing and harvesting of rice plants followed by, for example, the sowing and optional harvesting of soy bean, potatoes or any other suitable plant), thereby increasing the annual biomass production per acre (due to an increase in the number of times (say in a year) that any particular plant may be grown and harvested). An increase in growth rate may also allow for the cultivation of transgenic plants in a wider geographical area than their wild-type counterparts, since the territorial limitations for growing a crop are often determined by adverse environmental conditions either at the time of planting (early season) or at the time of harvesting (late season). Such adverse conditions may be avoided if the harvest cycle is shortened. The faster rate of growth may be determined by deriving various parameters from growth curves derived from growth experiments, such parameters may be: T-Mid (the time taken for plants to reach 50% of their maximal size) and T-90 (time taken for plants to reach 90% of their maximal size).

According to a preferred feature of the present invention, performance of the methods of the invention result in plants having modified growth rate. Therefore, according to the present invention, there is provided a method for increasing the growth rate of plants, which method comprises modulating expression in a plant of a B-type CDK and/or modulating activity and/or levels in a plant of a B-type CDK protein. An increase in growth rate is demonstrated in the examples by the reduced time taken for transgenic plants to reach maturity than control plants.

An increase in yield also encompasses a better performance of the plant under non-stress conditions as well as under stress conditions compared to wild-type plants. Plants typically respond to exposure to stress by growing more slowly. However, since the transgenic plants according to the present invention have increased yield and increased growth rate, it is apparent that transgenic plants will also grow faster during stress conditions than corresponding wild type plants also exposed to the same stress conditions. The stress conditions will typically be the everyday biotic and/or abiotic (environmental) stresses to which a plant may be exposed. Typical abiotic or environmental stresses include temperature stresses caused by atypical hot or cold/freezing temperatures; salt stress; water stress (drought or excess water). Abiotic stresses may also be caused by chemicals. Biotic stresses as typically those stresses caused by pathogens, such as bacteria, viruses, fungi and insects.

“Modified architecture” as defined herein includes any a change in the appearance of any one or more of the leaves, shoots, stems, tillers, inflorescence (for monocotyledonous and dicotyledonous plants), panicles, pollen, ovule, seed, embryo, endosperm, seed coat and aleurone.

According to a preferred feature of the present invention, performance of the methods according to the present invention result in plants having modified architecture. The modified architecture is manifested by at least one of: an increase in aboveground area, an increase in the number of panicles and an increase in height. Therefore, according to the present invention, there is provided a method for modifying the architecture of plants, comprising modulating expression in a plant of a B-type CDK nucleic acid/gene and/or modulating activity and/or levels in a plant of a B-type CDK protein.

The methods according to the present invention result in plants having modified growth characteristics, as described hereinbefore. These advantageous growth characteristics may be combined with other economically advantageous traits, such as further yield-enhancing traits, tolerance to various stresses, traits modifying various architectural features and/or biochemical and/or physiological features.

According to a further embodiment of the present invention, the use of a B-type CDK is provided. For example, B-type CDKs may be used in breeding programmes. The nucleic acid sequence may be on a chromosome, or a part thereof, comprising at least the nucleic acid sequence encoding the B-type CDK protein and preferably also one or more related family members. In an example of such a breeding programme, a DNA marker is identified which may be genetically linked to a gene capable of modulating expression of a nucleic acid encoding a B-type CDK protein in a plant, which gene may be a gene encoding the B-type CDK protein itself or any other gene capable of directly or indirectly influencing expression of a B-type CDK gene and/or activity and/or levels of a B-type CDK protein. This DNA marker may then used in breeding programs to select plants having altered growth characteristics.

Allelic variants of B-type CDKs may be used in particular conventional breeding programmes, such as in marker-assisted breeding. Such breeding programmes sometimes require the introduction of allelic variations in the plants by mutagenic treatment of a plant. One suitable mutagenic method is EMS mutagenesis. Identification of allelic variants then takes place by, for example, PCR. This is followed by a selection step for selection of superior allelic variants of the sequence in question and which give rise to altered growth characteristics of a plant. Selection is typically carried out by monitoring growth performance of plants containing different allelic variants of the sequence in question, for example, different allelic variants of SEQ ID NO: 1. Monitoring growth performance can be done in a greenhouse or in the field. Further optional steps include crossing plants, in which the superior allelic variant was identified, with another plant. This could be used, for example, to make a combination of interesting phenotypic features. Allelic variants also encompass Single Nucleotide Polymorphisms (SNPs), as well as Small Insertion/Deletion Polymorphisms (INDELs). The size of INDELs is usually less than 100 bp). SNPs and INDELs form the largest set of sequence variants in naturally occurring polymorphic strains of most organisms.

The present invention also relates to use of a B-type CDK nucleic acid/gene and to use of a B-type CDK protein in modifying the growth characteristics of plants, preferably in modifying one or more of the following characteristics: increasing the area of a plant, increasing the number of first panicles, increasing plant height, increasing the number of seeds, increasing the number of filled seeds, increasing the total weight of seeds, increasing the growth rate, increasing the harvest index and increasing the thousand kernel weight (TKW).

The B-type CDK nucleic acid may be isolated from a monocotyledonous or dicotyledonous species, preferably from the family Brassicaceae, further preferably from Arabidopsis thaliana. The nucleic acid is preferably a class 1 B-type CDK, such as a class 1 B-type CDK selected from the examples of class 1 CDKs shown in FIG. 1, namely, CDK B1;1 from Arabidopsis thaliana, CDK B1;2 from Arabidopsis thaliana, CDKB1;1 from Lycopersicon esculentum (tomato), CDK B1;1 from Antirrhinum majus, CDK B1;1 from Medicago sativa (alfalfa) and CDK B1 from Dunaliella tertiolecta. Further preferably the class 1 B-type CDK is a CDK B1;1 from Arabidopsis thaliana or a CDK B1;2 from Arabidopsis thaliana. Alternatively, the nucleic acid is preferably a class 2 B-type CDK, such as a class 2 B-type CDK selected from the examples shown in FIG. 1, namely, a CDK B2;1 from Arabidopsis thaliana, a CDK B2;2 from Arabidopsis thaliana, a CDK B2;1 from Antirrhinum majus, a CDK B2;1 from Mesembryanthemum crassifolium, a CDK B2;1 from Medicago sativa, a CDK B2;1 from Lycopersicon esculentum and a CDK B1 from Oryza sativa. Further preferably the class 2 B-type CDK is a CDK B2;2 from Arabidopsis thaliana.

Most preferably the CDK B1;1 nucleic acid is as represented by SEQ ID NO: 1 or by a portion thereof, or by a nucleic acid sequence capable of hybridising therewith, and wherein the CDK B1;1 protein is as represented by SEQ ID NO: 2, or a homologue, derivative or active fragment thereof. Most preferably the CDK B1;2 nucleic acid is as represented by SEQ ID NO: 3 or by a portion thereof, or by a nucleic acid sequence capable of hybridising therewith, and wherein the CDK B1;2 protein is as represented by SEQ ID NO: 4, or a homologue, derivative or active fragment thereof. Most preferably the CDK B2;2 nucleic acid is as represented by SEQ ID NO: 5 or by a portion thereof, or by a nucleic acid sequence capable of hybridising therewith, and wherein the CDK B2;2 protein is as represented by SEQ ID NO: 6, or a homologue, derivative or active fragment thereof. Each of the CDK B1;1, CDKB1;2 and CDK B2;2 nucleic acids/proteins also encompass the variant nucleic acids and amino acids as described hereinbefore.

The present invention also relates to the use of a B-type CDK nucleic acid/gene and/or to the use of a B-type CDK protein as growth regulators. The B-type CDK nucleic acid sequences hereinbefore described and the B-type CDK amino acid sequences hereinbefore described are clearly useful in modifying the growth characteristics of plants. The sequences would therefore find use as growth regulators or growth stimulators. The present invention also provides a composition comprising a B-type CDK protein as hereinbefore described for the use as a growth regulator.

Conversely, the sequences according to the present invention may also be interesting targets for agrochemical compounds, such as herbicides. Accordingly, the present invention encompasses use of the aforementioned B-type CDK nucleic acids as targets for agrochemical compounds, such as herbicides.

### DESCRIPTION OF FIGURES

The present invention will now be described with reference to the following figures in which:

FIG. 1 is a phylogenetic tree showing the relationship of CDKs from various plants.

FIG. 2 is an enlarged view of the CDK A branch of the phylogenetic tree of FIG. 1.

FIG. 3 is a map of the binary vector for the expression in Oryza sativa of an Arabidopsis thaliana CDKB1;1 gene under the control of a putative beta-expansin promoter, EXPB8 (SEQ ID NO: 14).

FIG. 4 is a map of the binary vector for the expression in Oryza sativa of an Arabidopsis thaliana CDKB1;2 gene under the control of a GOS2 promoter (SEQ ID NO: 15).

FIG. 5 is a map of the binary vector for the expression in Oryza sativa of an Arabidopsis thaliana CDKB2;2 gene under the control of a GOS2 promoter (SEQ ID NO: 15).

FIG. 6 details examples of sequences useful in performing the methods according to the present invention.

FIG. 7 is a CLUSTAL W (1.82) multiple sequence alignment for some representative CDK B-type sequences. In bold is a motif found in B-type CDKs; underlined is a catalytic kinase domain and in italics is shown a T-loop activation kinase domain (Magyar et al., 1997 (Plant Cell 9 (2), 223-235)).

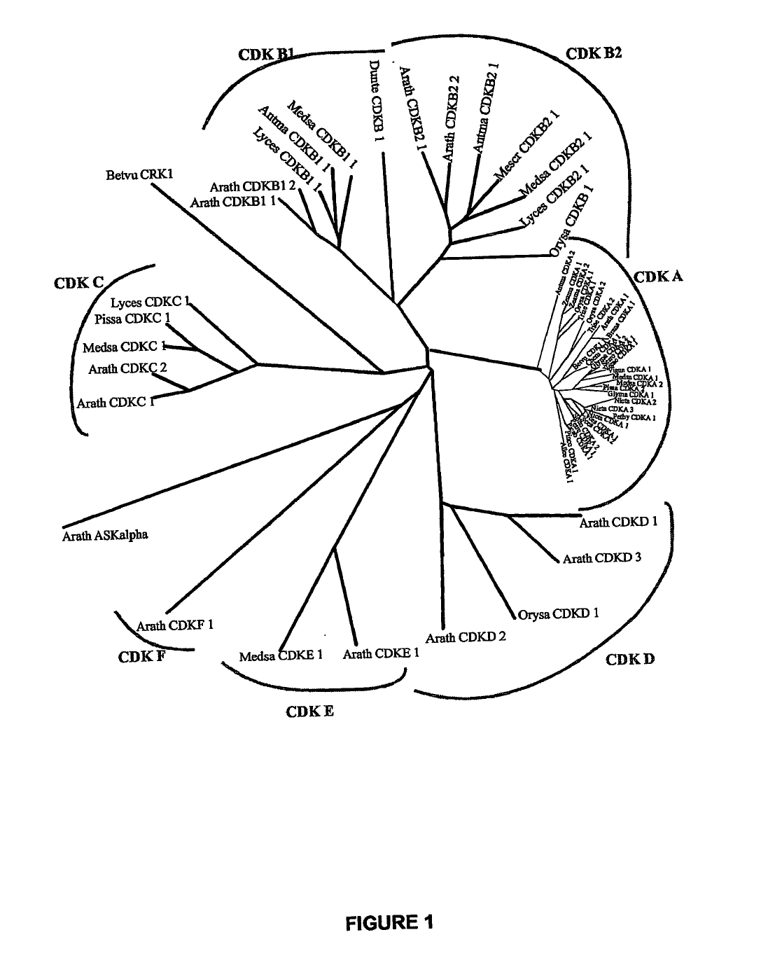

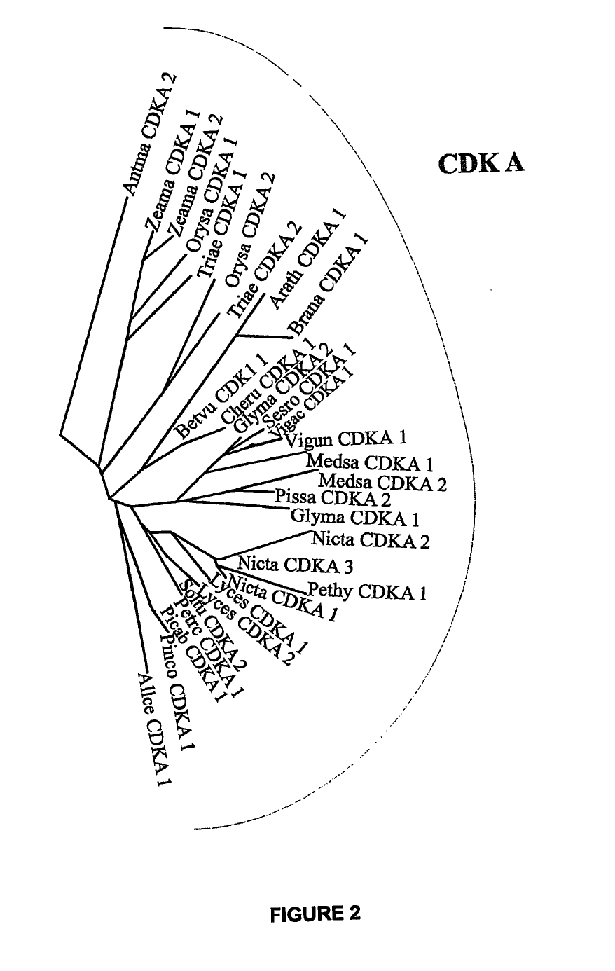

### EXAMPLES

The present invention will now be described with reference to the following examples, which are by way of illustration alone.

DNA manipulation: unless otherwise stated, recombinant DNA techniques are performed according to standard protocols described in (Sambrook (2001) Molecular Cloning: a laboratory manual, 3rd Edition Cold Spring Harbor Laboratory Press, CSH, New York) or in Volumes 1 and 2 of Ausubel et al. (1994), Current Protocols in Molecular Biology, Current Protocols. Standard materials and methods for plant molecular work are described in Plant Molecular Biology Labfase (1993) by R. D. D. Croy, published by BIOS Scientific Publications Ltd (UK) and Blackwell Scientific Publications (UK).

### Example 1

### Gene Cloning—CDK B1;1

The Arabidopsis CDK B1;1 was amplified by PCR using as a template an Arabidopsis thaliana seedling cDNA library (Invitrogen, Paisley, UK). After reverse transcription of RNA extracted from seedlings, the cDNAs were cloned into pCMV Sport 6.0. Average insert size of the bank was 1.5 kb and original number of clones was 1.59×10⁷ cfu. Original titer was determined to be 9.6×10⁵ cfu/ml and, after first amplification, 6×10¹¹ cfu/ml. After plasmid extraction, 200 ng of template was used in a 50 μl PCR mix. Primers prm0350 (sense, start codon in bold, AttB1 site in italic: 5′ GGGGACAAGTTTGTACAAAAAAGCAGGCTTCACAATGGAGAAGTACGAG AAGCTAGA 3′) and prm0351 (reverse, complementary, stop codon in bold, AttB2 site in italic: 5′ GGGGACCACTTTGTACAAGAAAGCTGGGTTCAGAACTGAGACTTGTCAAGG 3′), which include the AttB sites for Gateway recombination, were used for PCR amplification. PCR was performed using Hifi Taq DNA polymerase in standard conditions. A PCR fragment of 930 bp was amplified and purified also using standard methods. The first step of the Gateway procedure, the BP reaction, was then performed, during which the PCR fragment was recombined in vivo with the pDONR201 plasmid to produce, according to Gateway terminology, an “entry clone”, p0438. Plasmid pDONR201 was purchased from Invitrogen, as part of the Gateway® technology.

### Example 2

### Vector Construction—CDK B1;1

The entry clone p0438 was subsequently used in an LR reaction with p3169, a destination vector used for Oryza sativa transformation. This vector contains as functional elements within the T-DNA borders: a plant selectable marker, a plant screenable marker and a Gateway cassette intended for LR in vivo recombination with the sequence of interest already cloned in the entry clone. A putative beta-expansin promoter for expression in young expanding tissue is located upstream of this Gateway cassette.

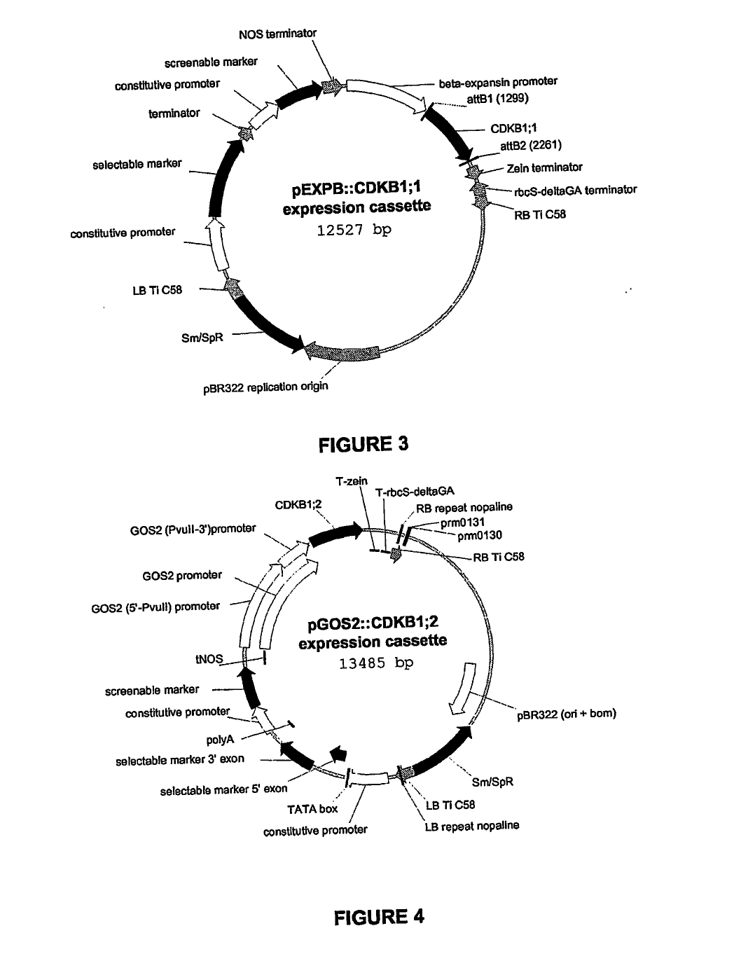

After the LR recombination step, the resulting expression vector as shown in FIG. 3 (CDK B1;1: beta-expansin—overexpression) was transformed into Agrobacterium and subsequently to Oryza sativa plants. Transformed rice plants were allowed to grow to and then examined for various parameters as described in Example 7.

### Example 3

### Gene Cloning—CDK B1;2

The Arabidopsis CDK B1;2 was amplified by PCR using as a template an Arabidopsis thaliana seedling cDNA library (Invitrogen, Paisley, UK). After reverse transcription of RNA extracted from seedlings, the cDNAs were cloned into pCMV Sport 6.0. Average insert size of the bank was 1.5 kb, and original number of clones was of 1.59×10⁷ cfu. Original titer was determined to be 9.6×10⁵ cfu/ml, after first amplification of 6×10¹¹ cfu/ml. After plasmid extraction, 200 ng of template was used in a 50 μl PCR mix. Primers prm439 (sense, start codon in bold, AttB1 site in italic: 5′ GGGGACAAGTTTGTACAAAAAAGCAGGCTTCACAATGGAGAAATACG AGAAGCTC 3′) and prm440 (reverse, complementary, stop codon in bold, AttB2 site in italic: 5′ GGGGACCACTTIGTACAAGAAAGCTGGGTGGTCAGAACTGAGATTTGTC 3′), which include the AttB sites for Gateway recombination, were used for PCR amplification. PCR was performed using Hifi Taq DNA polymerase in standard conditions. A PCR fragment of 936 bp was amplified and purified also using standard methods. The first step of the Gateway procedure, the BP reaction, was then performed, during which the PCR fragment was recombined in vivo with the pDONR201 plasmid to produce, according to the Gateway terminology, an “entry clone”, p538. Plasmid pDONR201 was purchased from Invitrogen, as part of the Gateway® technology

### Example 4

### Vector Construction CDK B1;2

The entry clone p538 was subsequently used in an LR reaction with p640, a destination vector used for Oryza sativa transformation. This vector contains as functional elements within the T-DNA borders: a plant selectable marker; a plant screenable marker; and a Gateway cassette intended for LR in vivo recombination with the sequence of interest already cloned in the entry clone. A GOS2 promoter for upregulation was located upstream of this Gateway cassette.

After the LR recombination step, the resulting expression vector as shown in FIG. 4 (CDK B1;2: GOS 2—overexpression) was transformed into Agrobacterium and subsequently to Oryza sativa plants. Transformed rice plants were allowed to grow to and then examined for various parameters as described in Example 7.

### Example 5

### Gene Cloning—CDK B2;2

The Arabidopsis CDKB2;2 was amplified by PCR using as a template an Arabidopsis thaliana seedling cDNA library (Invitrogen, Paisley, UK). After reverse transcription of RNA extracted from seedlings, the cDNAs were cloned into pCMV Sport 6.0. Average insert size of the bank was 1.5 kb, and original number of clones was of 1.59×10⁷ cfu. Original titer was determined to be 9.6×10⁵ cfu/ml, after first amplification of 6×10¹¹ cfu/ml. After plasmid extraction, 200 ng of template was used in a 50 μl PCR mix. Primers prm2213 (sense, start codon in bold, AttB1 site in italic: 5′ GGGGACAAGTTTGTACAAAAAAGCAGGCTTCACAATGGACAACAATGG AGTTAA 3′) and prm2214 (reverse, complementary, stop codon in bold, AttB2 site in italic: 5′ GGGGACCACTTTGTACAAGAAAGCTGGGTTCAGAGAGAGGACTTGTCAG 3′), which include the AttB sites for Gateway recombination, were used for PCR amplification. PCR was performed using Hifi Taq DNA polymerase in standard conditions. A PCR fragment of 948 bp was amplified and purified also using standard methods. The first step of the Gateway procedure, the BP reaction, was then performed, during which the PCR fragment was recombined in vivo with the pDONR201 plasmid to produce, according to Gateway terminology, an “entry clone”, p2660. Plasmid pDONR201 was purchased from Invitrogen, as part of the Gateway® technology.

### Example 6

### Vector Construction—CDK B2;2

The entry clone p2660 was subsequently used in an LR reaction with p640, a destination vector used for Oryza sativa transformation. This vector contains as functional elements within the T-DNA borders: a plant selectable marker; a plant screenable marker; and a Gateway cassette intended for LR in vivo recombination with the sequence of interest already cloned in the entry clone. A pGOS2 promoter for overexpression was located upstream of this Gateway cassette.

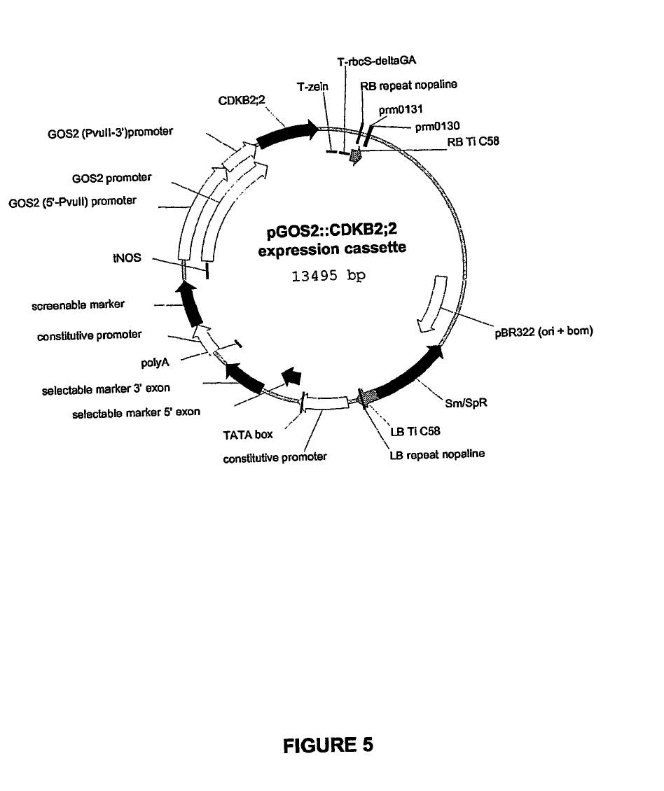

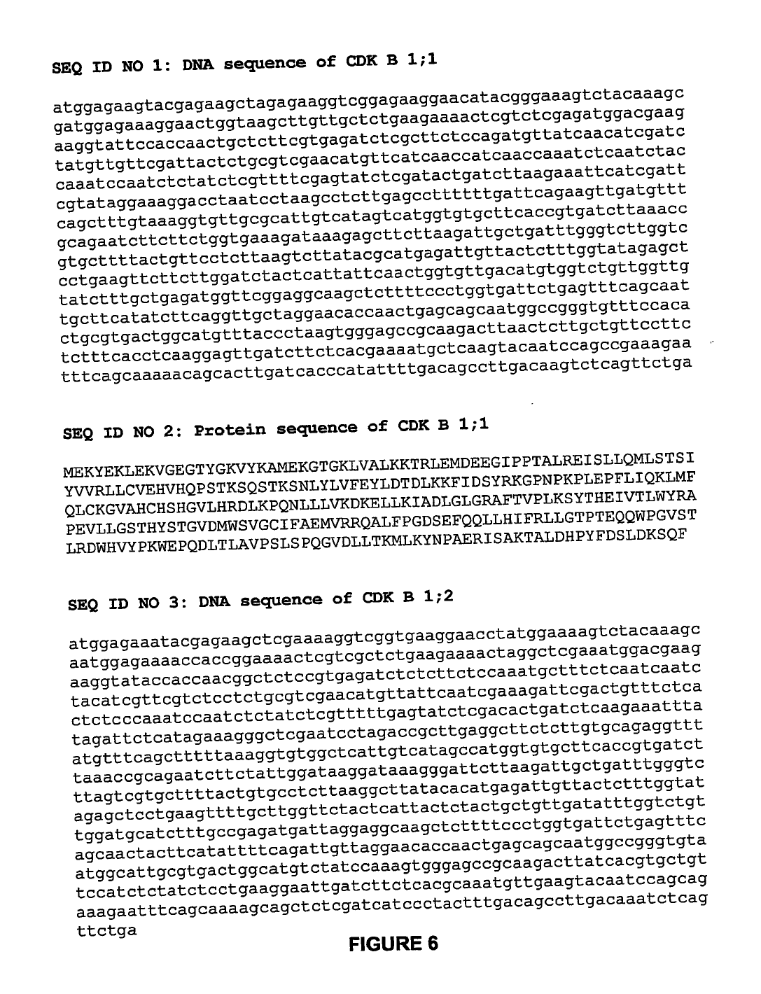

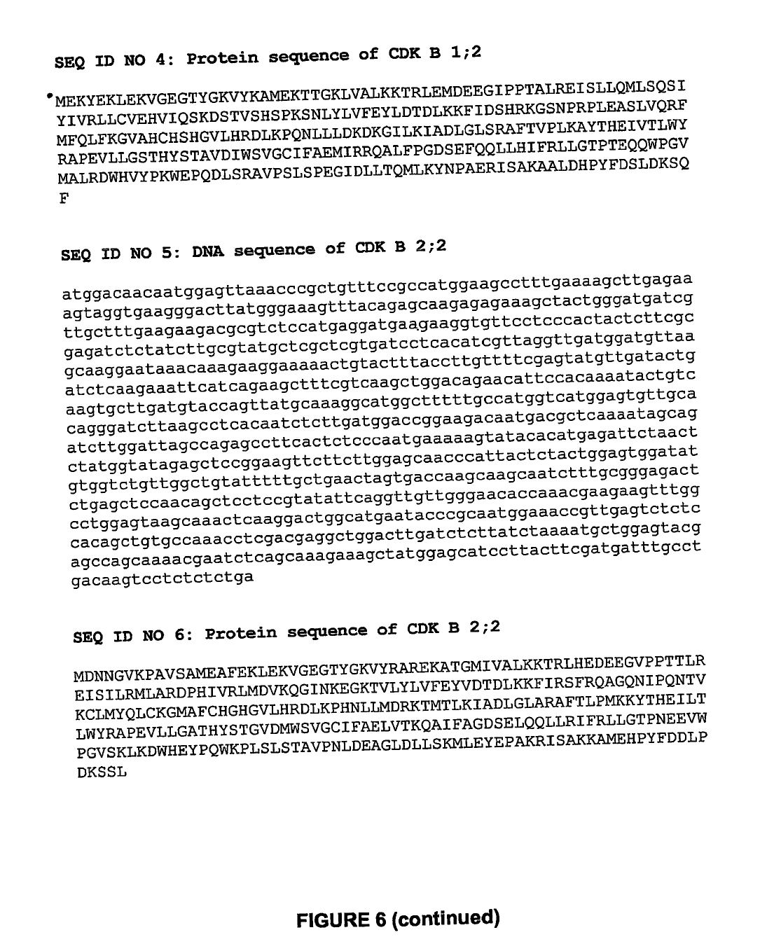

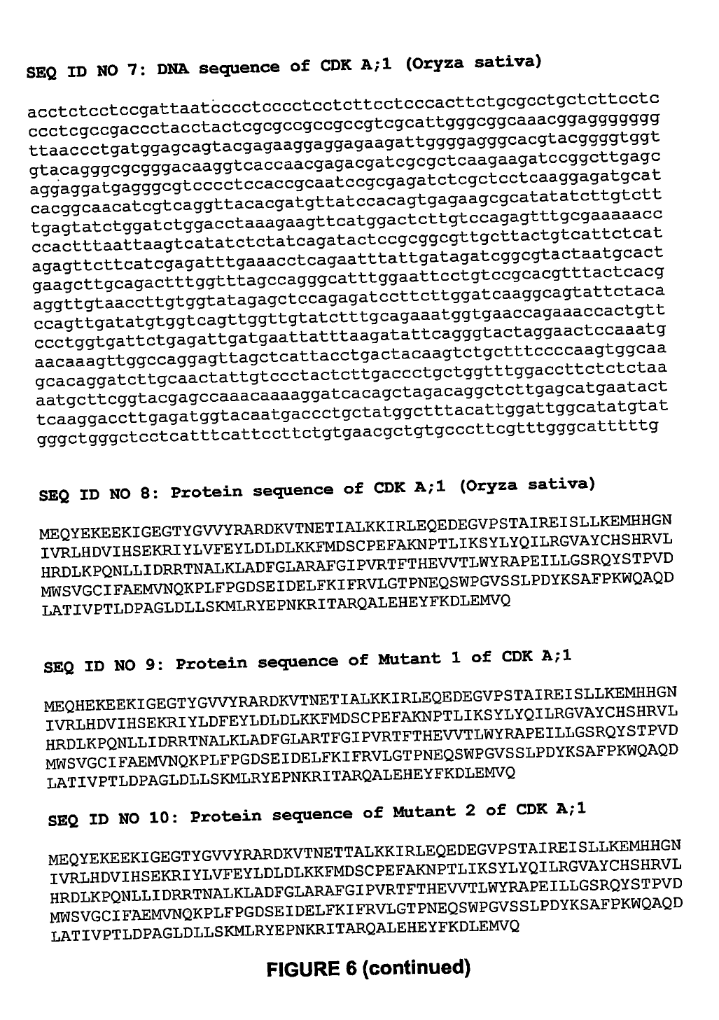

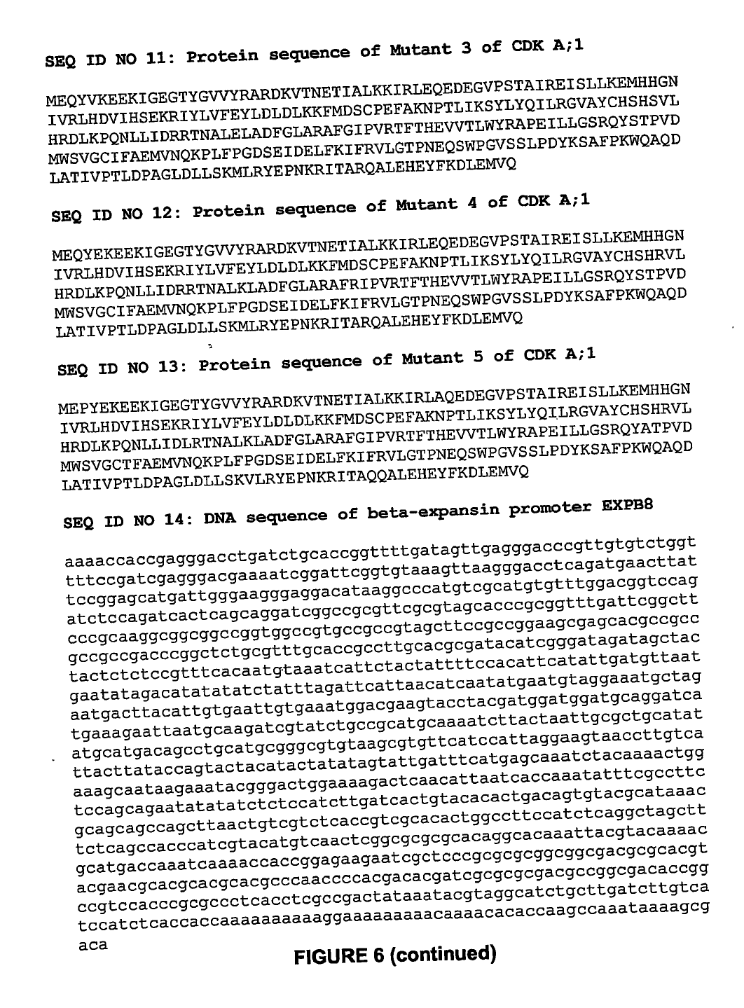

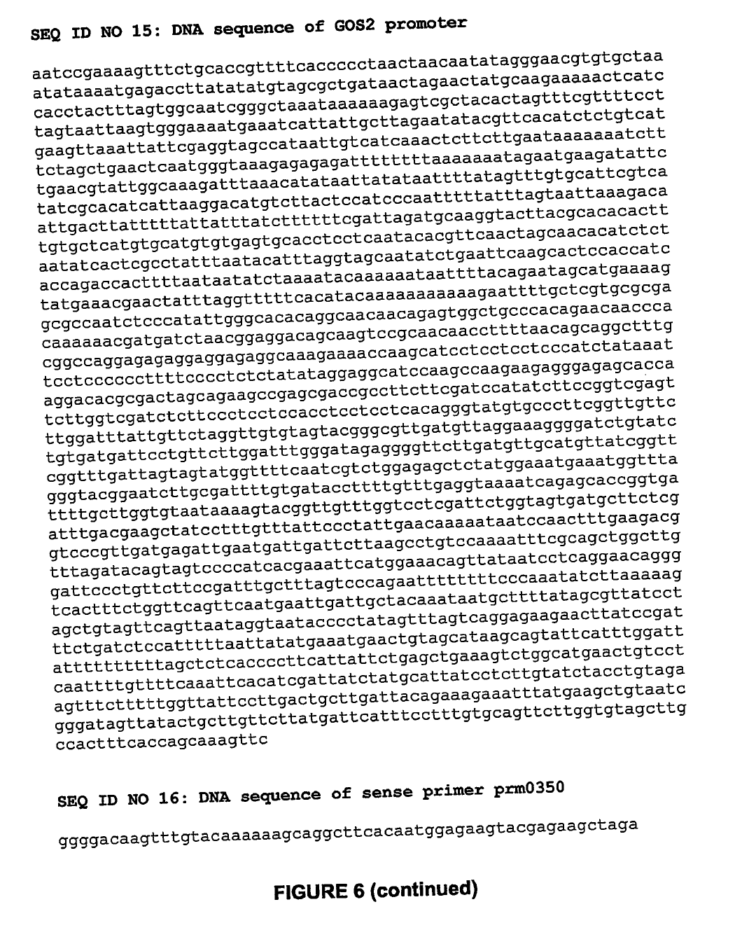

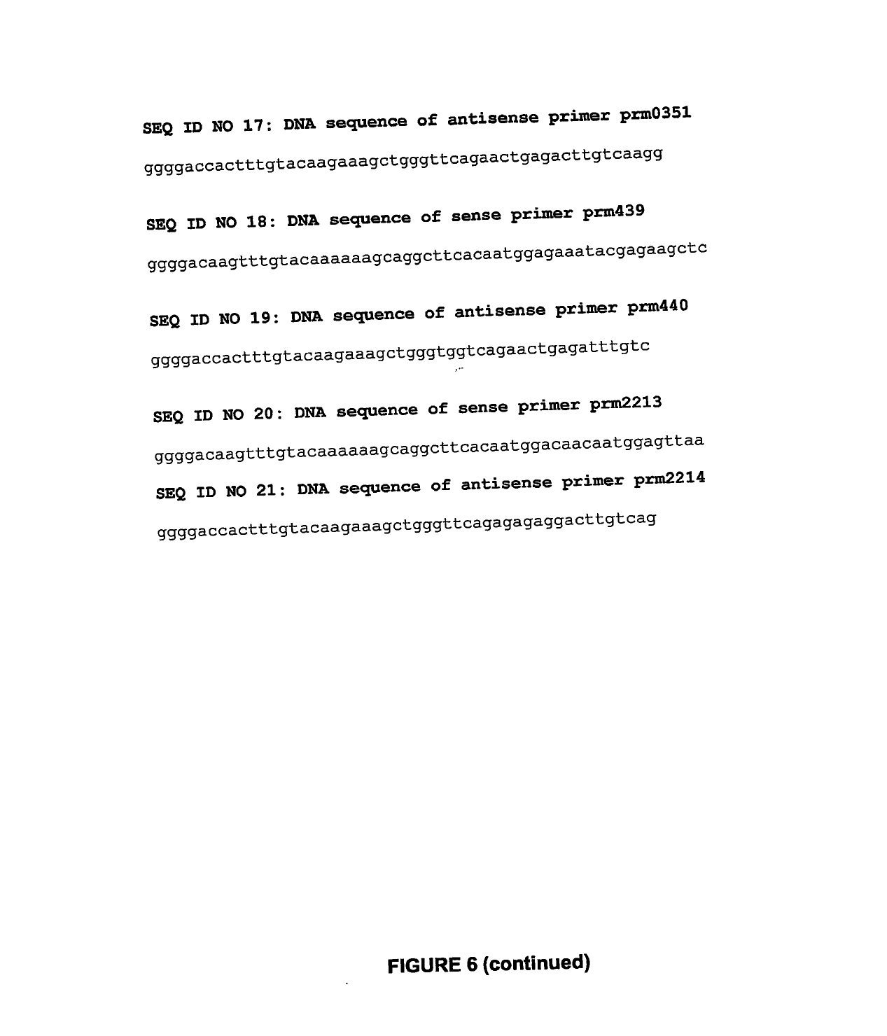

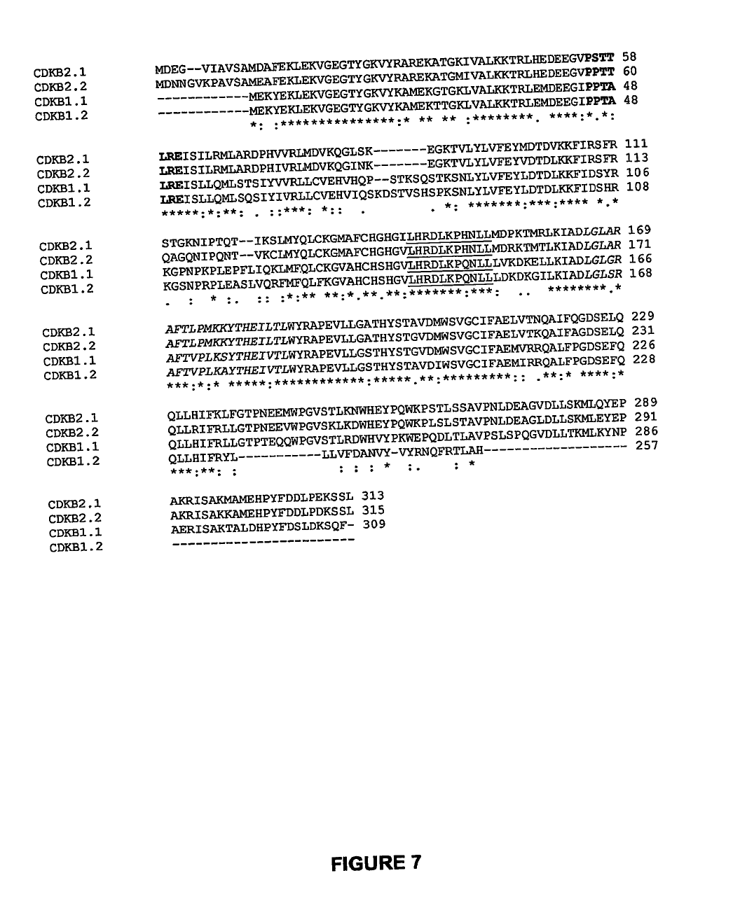

After the LR recombination step, the resulting expression vector as shown in FIG. 5 (CDK B2;2:

GOS2—overexpression) was transformed into Agrobacterium and subsequently into Oryza sativa plants. Transformed rice plants were allowed to grow to and then examined for various parameters as described in Example 7.

### Example 7

### Evaluation and Results

Approximately 15 to 20 independent T0 rice transformants were generated. The primary transformants were transferred from tissue culture chambers to a greenhouse for growing and harvest of T1 seed. 5 events, of which the T1 progeny segregated 3:1 for presence/absence of the transgene, were retained. For each of these events, approximately 10 T1 seedlings containing the transgene (hetero- and homo-zygotes), and approximately 10 T1 seedlings lacking the transgene (nullizygotes), were selected by monitoring visual marker expression.

Statistical Analysis: T-Test and F-Test

A two factor ANOVA (analysis of variants) was used as statistical model for the overall evaluation of plant phenotypic characteristics. An F-test was carried out on all the parameters measured, for all of the plants of all of the events transformed with the gene of interest. The F-test was carried out to check for an effect of the gene over all the transformation events and to determine the overall effect of the gene or “global gene effect”. Significant data, as determined by the value of the F-test, indicates a “gene” effect, meaning that the phenotype observed is caused by more than the presence or position of the gene. In the case of the F-test, the threshold for significance for a global gene effect is set at a 5% probability level.

To check for an effect of the genes within an event, i.e., for a line-specific effect, a t-test was performed within each event using data sets from the transgenic plants and the corresponding null plants. “Null plants” or “Null segregants” are the plants treated in the same way as the transgenic plant, but from which the transgene has segregated. Null plants can also be described as the homozygous negative transformants. The threshold for significance for the t-test is set at 10% probability level. Within one population of transformation events, some events can be under or above this t-test threshold. This is based on the hypothesis that a gene might only have an effect in certain positions in the genome, and that the occurrence of this position-dependent effect is not uncommon. This kind of gene effect may also be referred to as a “line effect of a gene”. The p-value is obtained by comparing the t-value to the t-distribution or alternatively, by comparing the F-value to the F-distribution. The p-value stands for the probability that the null hypothesis (null hypothesis being “there is no effect of the transgene”) is correct.

7.1 Vegetative Growth Measurements:

The selected T1 plants (approximately 10 with the transgene and approximately 10 without the transgene) were transferred to a greenhouse. Each plant received a unique barcode label to link unambiguously the phenotyping data to the corresponding plant. The selected T1 plants were grown on soil in 10 cm diameter pots under the following environmental settings: photoperiod=11.5 h, daylight intensity=30,000 lux or more, daytime temperature=28° C. or higher, night time temperature=22° C., relative humidity=60-70%. Transgenic plants and the corresponding nullizygotes were grown side-by-side at random positions. From the stage of sowing until the stage of maturity each plant was passed several times through a digital imaging cabinet and imaged. At each time point digital images (2048×1536 pixels, 16 million colours were taken of each plant from at least 6 different angles. The parameters described below were derived in an automated way from all the digital images of all the plants, using image analysis software.

In the tables of results below, each row corresponds to one event. The numeric difference between the positive plants and the negative plants is given (dif) as well as the percentage difference between these plants (% dif). P-value stands for the probability produced by the t-test for each plant line. The last row shows average numbers for all events. In this row, the p-value is the p-value from the F-test.

(a) Aboveground Plant Area

Plant aboveground area was determined by counting the total number of pixels from aboveground plant parts discriminated from the background. This value was averaged for the pictures taken on the same time point from the different angles and was converted to a physical surface value expressed in square mm by calibration. Experiments show that the aboveground plant area measured this way correlates with the biomass of plant parts above ground.

TABLE 2
T1 Aboveground plant area - CDK B1; 1
T1 Aboveground plant area - CDK B1; 1

| Line | % dif | p-value |
|---|---|---|
| 1 | −3 | 0.7936 |
| 2 | 11 | 0.4897 |
| 3 | 53 | 0.0011 |
| 4 | 10 | 0.4096 |
| 5 | −2 | 0.8065 |
| Overall | 10 | 0.0729 |

As shown in Table 1, line 3 gave a significant increase in the aboveground area for transgenic plants relative to control plants, with a p-value from the t-test of 0.0011. Lines 2 and 4 also showed an increase in the aboveground plant area relative to that of control plants. An overall increase of 10% was seen in the aboveground area of transgenic plants compared to control plants.

TABLE 3
T1 Aboveground plant area - CDK B1; 2
T1 Aboveground plant area - CDK B1; 2

| Line | % dif | p-value |
|---|---|---|
| 10 | −5 | 0.6726 |
| 11 | 34 | 0.0002 |
| 12 | 19 | 0.0573 |
| 13 | −6 | 0.4359 |
| Overall | 11 | 0.0178 |

As shown in Table 2, lines 11 and 12 gave a significant increase in aboveground plant area with respective p-values from the t-test of 0.0002 and 0.0573. An overall gene effect was also apparent from a p-value of 0.0178 from the F-test. An overall increase of 11% was seen in the aboveground area of transgenic plants compared to control plants. Line 11 was also confirmed in the T2 generation (see Table 4 below) with a 30% increase compared to corresponding nullizygotes and with a p-value from the t test of 0.0002.

TABLE 4
T2 Aboveground plant area - CDK B1; 2
T2 Aboveground plant area - CDK B1; 2

| Line | % dif | p-value |
|---|---|---|
| 13 | 5 | 0.5379 |
| 12 | −8 | 0.2791 |
| 11 | 30 | 0.0002 |
| Overall | 9 | 0.0508 |

(b) Plant Height

Plant height was determined by the distance between the horizontal lines going through the upper pot edge and the uppermost pixel corresponding to a plant part above ground. This value was averaged for the pictures taken on the same time point from the different angles and was converted, by calibration, to a physical distance expressed in mm. Experiments showed the plant height measured this way correlates with plant height measured manually with a ruler.

TABLE 5
T1 Height - CDK B1; 1
T1 Height - CDK B1; 1

| Line | % dif | p-value |
|---|---|---|
| 1 | −5 | 0.3466 |
| 2 | 1 | 0.8181 |
| 3 | 19 | 0.0045 |
| 4 | 6 | 0.3358 |
| 5 | −2 | 0.7003 |
| Overall | 3 | 0.2693 |

The results are shown in Table 5. As shown, line 3 showed a significant increase in plant height relative to corresponding control plants (with a p value from the t-test of 0.0045).

TABLE 6
T1 Height - CDK B1; 2
T1 Height - CDK B1; 2

| Line | % Diff | p-value |
|---|---|---|
| 13 | −9 | 0.1222 |
| 12 | 10 | 0.1084 |
| 11 | 9 | 0.1022 |
| 10 | −4 | 0.5187 |
| Overall | 2 | 0.5361 |

TABLE 7
T2 Height - CDK B1; 2
T2 Height - CDK B1; 2

| Line | % dif | p-value |
|---|---|---|
| 11 | 9 | 0.019 |
| 12 | −3 | 0.4815 |
| 13 | 5 | 0.2379 |
| Overall | 4 | 0.0945 |

Table 6 shows an increase in height in the T1 generation for lines 11 and 12. As shown in Table 7, line 11 showed a significant increase in height in T2 generation plants relative to control plants and gave a p value from the t-test of 0.019.

7.2 Seed-Related Parameter Measurements

The mature primary panicles were harvested, bagged, barcode-labelled and then dried for three days in the oven at 37° C. The panicles were then threshed and all the seeds were collected and counted. The filled husks were separated from the empty ones using an air-blowing device. The empty husks were discarded and the remaining fraction was counted again. The filled husks were weighed on an analytical balance. This procedure resulted in the set of seed-related parameters described below.

(c) Total Seed Number Per Plant

This was measured by counting the number of husks harvested from a plant.

TABLE 8
T1 Total seed number - CDK B1; 1
T1 Total Seed Number - CDK B1; 1

| Line | % dif | p-value |
|---|---|---|
| 1 | 10 | 0.4842 |
| 2 | 10 | 0.6685 |
| 3 | 80 | 0.0002 |
| 4 | 3 | 0.8577 |
| 5 | −10 | 0.4176 |
| Overall | 12 | 0.081 |

The results are shown in Table 8 above. As shown, line 3 gave a significant increase in the total number of seeds produced by transgenic plants relative to the total number of seeds produced by control plants (with a p value from the t-test of 0.0002).

TABLE 9
T2 Total seed number - CDK B1; 2
T2 Total Seed Number - CDK B1; 2

| Line | % dif | p-value |
|---|---|---|
| 11 | 32 | 0.0047 |
| 12 | −16 | 0.1356 |
| 13 | −0 | 0.9856 |
| Overall | 5 | 0.4425 |

As shown in Table 9, line 11 showed a significant increase (with a p value from the t-test of 0.0047) in the total number of seeds of transgenic plants relative to the total number of seeds of control plants.

(d) Number of Filled Seeds

The number of filled seeds was determined by counting the number of filled husks that remained after the separation step.

TABLE 10
T1 Number of filled seeds - CDK B1; 1
T1 Number of Filled Seeds - CDK B1; 1

| Line | % dif | p-value |
|---|---|---|
| 1 | 20 | 0.3353 |
| 2 | 4 | 0.881 |
| 3 | 67 | 0.0072 |
| 4 | −10 | 0.6329 |
| 5 | −4 | 0.8055 |
| Overall | 12 | 0.2026 |

The results are shown in Table 10 above. As shown, line 3 showed a significant increase in the number of filled seeds relative to that of control plants (with a p value of the t test of 0.0072).

TABLE 11
T1 Number of filled seeds - CDK B1; 2
T1 Number of filled seeds - CDK B1; 2

| Line | % dif | p-value |
|---|---|---|
| 10 | 10 | 0.7578 |
| 11 | 56 | 0.0091 |
| 12 | −18 | 0.3294 |
| 13 | −6 | 0.7196 |
| Overall | 14 | 0.3805 |

TABLE 12
T2 Number of filled seeds - CDK B1; 2
T2 Number of filled seeds - CDK B1; 2

| Line | % Diff | p-value |
|---|---|---|
| 13 | −2 | 0.8861 |
| 12 | −23 | 0.1299 |
| 11 | −45 | 0.0013 |
| Overall | 8 | 0.3391 |

As shown in Table 11, line 11 showed an increase in the number of filled seeds relative to control plants with a p value from the t-test of 0.0091. An overall difference of 14% was observed for the number of filled seeds of transgenic plants relative to the number of filled seeds for corresponding control plants. The results of the T2 generation are shown in Table 12, with line 11 performing particularly well.

(e) Total Seed Yield Per Plant

The total seed yield was measured by weighing all filled husks harvested from a plant.

TABLE 13
T1 Total weight of seeds - CDK B1; 1
T1 Total Weight Seeds - CDK B1; 1

| Line | % dif | p-value |
|---|---|---|
| 1 | 21 | 0.3578 |
| 2 | 28 | 0.4186 |
| 3 | 75 | 0.005 |
| 4 | −9 | 0.6551 |
| 5 | −3 | 0.8182 |
| Overall | 16 | 0.1096 |

The results are shown in Table 13 above. As shown, line 3 showed a significant increase (with a p value from the t test of 0.005) in the total weight of seeds of transgenic plants relative to the total weight of the seeds of corresponding non-transgenic plants. An overall increase of 16% was observed for the total weight of the seeds of transgenic plants verses the total weight of the seeds of control plants.

TABLE 14
T1: Total weight of seeds - CDK B1; 2
T1 Total Weight Seeds - CDK B1; 2

| Line | % dif | p-value |
|---|---|---|
| 10 | 2 | 0.7587 |
| 11 | 46 | 0.0139 |
| 12 | −13 | 0.4276 |
| 13 | −7 | 0.6736 |
| Overall | 11 | 0.5841 |

As shown in Table 14, line 11 showed a significant increase in the total weight of the seeds of transgenic plants relative to the total weight of seeds of control plants with a p value from the t-test of 0.0139. The results of the T2 generation are given in Table 15 below, with line 11 performing particularly well.

TABLE 15
T2: Total weight of seeds - CDK B1; 2
T2 Total Weight Seeds - CDK B1; 2

| Line | % Diff | p-value |
|---|---|---|
| 13 | −6 | 0.7393 |
| 12 | −25 | 0.1509 |
| 11 | 54 | 0.0015 |
| Overall | 8 | 0.3694 |

(f) Harvest Index of Plants

The present invention is defined as the ratio between the total seed yield and the above ground area (mm²), multiplied by a factor 106.

TABLE 16
T1 Harvest Index - CDK B1; 1
T1 Harvest Index - CDK B1; 1

| Line | % dif | p-value |
|---|---|---|
| 1 | 10 | 0.5097 |
| 2 | −2 | 0.8972 |
| 3 | 45 | 0.0201 |
| 4 | −10 | 0.5009 |
| 5 | 0 | 0.9868 |
| Overall | 6 | 0.3644 |

The results are shown in Table 16 above. As shown, line 3 showed an increased harvest index for transgenic plants relative to the harvest index of control plants (with a p value from the t-test of 0.0201).

(g) Thousand Kernel Weight

Thousand Kernel Weight (TKW): this parameter is extrapolated from the number of filled seeds counted and their total weight.

TABLE 18
T1: Thousand Kernel Weight (TKW) - CDK B1; 2
T2 TKW - CDK B1; 2

| Line | % Diff | p-value |
|---|---|---|
| 13 | −1 | 0.8261 |
| 12 | 5 | 0.1968 |
| 11 | −7 | 0.0648 |
| 10 | −4 | 0.3081 |
| Overall | −2 | 0.3622 |

TABLE 19
T2: Thousand Kernel Weight (TKW) - CDK B1; 2
T2 TKW - CDK B1; 2

| Line | % dif | p-value |
|---|---|---|
| 11 | 14 | 0.0347 |
| 12 | 1 | 0.8554 |
| 13 | −5 | 0.2525 |
| Overall | 3 | 0.4471 |

The results of the T1 generation are shown in Table 18. Table 19 gives the results of the T2 generation. As shown, Line 11 gave a significant increase in the TKW of transgenic plants relative to the TKW of control plants with a p value from the t-test of 0.0347.

(h) Cycle Time—CDK B2:2

Weekly plant area measurements were modelled to obtain a growth curve for each plant. Plant area (in mm²) was plotted against time (in days) and from the resultant growth curve the following parameters were calculated.

TABLE 20
T1 Growth rate - CDK B2; 2
T1 Growth Rate (Total Area Cycle Time) - CDK B2; 2

| Line | % dif | p-value |
|---|---|---|
| 20 | −3 | 0.2143 |
| 21 | −4 | 0.0749 |
| 22 | 3 | 0.2296 |
| 23 | 4 | 0.0582 |
| 24 | 1 | 0.4851 |
| 25 | −0 | 0.9693 |
| 26 | −1 | 0.5318 |
| 27 | −4 | 0.0255 |
| Overall | −1 | 0.1062 |

The results are shown in Table 13 above. As shown, lines 21 and 27 gave significant increases in the growth rate of transgenic plants relative to the growth rate of control plants, with increases of 4 days observed in both cases.

### Example 8

### Transgenic Corn Expressing a B-type CDK

A B type CDK is cloned under control of a young expanding tissue-preferred or a constitutive promoter in a plant transformation vector suitable for Agrobacterium-mediated corn transformation. Vectors and methods for corn transformation are selected from those described in any of: EP0604662, EP0672752, EP0971578, EP0955371, EP0558676, Ishida et al. (Nat. Biotechnol. 1996 June; 14(6): 745-50); and Frame et al. (Plant Physiol. 2002 May; 129(1): 13-22)

Transgenic plants made by these methods are grown in the greenhouse for T1 seed production. Inheritability and copy number of the transgene is checked by quantitative real-time PCR and Southern blot analysis. Expression levels of the transgene are determined by reverse PCR and Northern analysis. Transgenic lines with single copy insertions of the transgene and with varying levels of transgene expression are selected for T2 seed production.

Progeny seeds are germinated and grown in a greenhouse in conditions adapted for maize (16:8 photoperiod, 26-28° C. daytime temperature and 22-24° C. night time temperature) as well under water-deficient, nitrogen-deficient, and excess NaCl conditions. In the case of selfing, null segregants from the same parental line, as well as wild type plants of the same cultivar are used as controls. The progeny plants resulting from the selfing or the crosses are evaluated for different biomass and growth parameters, including plant height, stem thickness, number of leaves, total above ground area, leaf greenness, time to maturity, flowering time, ear number, harvesting time. The seeds of these lines are also evaluated for changes in various parameters, such as grain size, total grain yield per plant, and grain quality (starch content, protein content and oil content).

Lines that are most significantly improved compared to corresponding control lines are selected for further field-testing and marker-assisted breeding, with the objective of transferring the field-validated transgenic traits into commercial germplasm. The testing of maize for growth and yield-related parameters in the field is conducted using well-established protocols. Similarly, introgressing specific loci (such as transgene containing loci) from one germplasm into another is also conducted using well-established protocols.

### Example 9

### Identification of Mutant B-Type CDKs

All molecular biology experiments were performed following standard procedures. Gateway destination vectors were amplified using Escherishia coli DB3.1 strain (F−, gyrA462, endA-, delta (sr1-recA), mcrB, mrr, hsdS20 (rB−, mB−), supE44, ara14, galK2, lacY1, proA2, rpsL20 (Smr), xyl5, lambda-, leu, mtl1). Other constructs were amplified using E. coli DH5-alpha strain (F−, phi80dlacZDelta M15, Delta (lacZYA-argF), U169, deoR, recA1, hadR17 (rk−,mk+), gal-, phoA, supE44, Lambda-, thi-1, gyrA96m, relA1).

9.0: Strains and Media Used

Saccharomyces cerevisiae CDK mutant strain US102 (MATa; leu2-3; ura3; trp1-1; his3-11; ade2-1; can1-100; cdc2B-1N) was used for complementation studies. Yeast cells were cultivated on rich medium (YPG-gal: yeast extract 0.5% w/v; peptone 0.5% v/w; galactose 1% w/v; raffinose 1% w/v, agar 2% w/v) or on synthetic medium (YNB: yeast nitrogen base without amino-acids (Difco, Becton Dickinson, Sparks, Madison, USA) 0.7% w/v, agar 2% w/v) supplemented with appropriate sugars and the adequate amino-acid drop-out mixture (Clontech, Palo-Alto, Calif., USA). Competent yeast cells were prepared and used for transformation using the Frozen-EZ Yeast Transformation II kit (ZymoResearch, Orange, Calif., USA) following the instructions of the manufacturer.

9.1: Two-Hybrid Vector Construction

pAD-Gal4 2.1 and pBD-gal4-cam (Stratagene, La Jolla, Calif., USA) were cut with Sma I, dephosphorylated, ligated to the Gateway cassette C (Life Technologies, Invitrogen Ltd, Paisley, UK) and introduced into Escherishia coli DB3.1. The orientation of the cassette was checked. The resulting vectors, pGW-AD and pGW-BD, contained the AttR1 site of the gateway cassette fused respectively to the GAL4 AD or BD domains, thus allowing insertion of coding sequences in-frame to the GAL4 AD or BD domains using the Gateway procedure (Life Technologies, Invitrogen Ltd, Paisley, UK).

Oriza sativa cdc2-1 (CDK A;1) cyclin dependent kinase full-length cDNA (accession number X60374, see also SEQ ID NOs 7 and 8) was amplified using AttB sites containing primers (Cdc2-1-AttB1: GGGGACAAGTTTGTACAAAAAAGCAGGCTTCACAATGGAGCAGTACGAG AAGGAGGAG, cdc2-1-AttB2: GGGGACCACTTTGTACAAGAAAGCTGGGTCCCCTGTCAT TGTACCATCTCAAG). The PCR products were introduced into pDONR201 (Life Technologies, Invitrogen Ltd, Paisley, UK) using the Gateway BP procedure. The resulting entry clones, pDONR-cdc2-1, were used to transfer the cdc2 coding sequences into the pGW-AD and pGW-BD vectors via the gateway LR reaction. The resulting plasmids, pAD-cdc2-1 and pBD-cdc2-1, contained the rice CDK A;1 coding sequences fused to the GAL4 AD or BD domains.

Protein-protein interactions were investigated following a mating 2-hybrid procedure using PJ69-4A (MATa, ura3-52, his3-200, ade2Δ, trp1901, leu2-3112, gal4Δ, gal80Δ, LYS2::GAL1 HIS3, ade2::GAL2-ADE2, met2::GAL7-lacZ. James et al., 1996) and PJ69-4alpha (MATalpha, ura3-52, his3-200, ade2Δ, trp1901, leu2-3112, gal4Δ, gal80Δ, LYS2::GAL1HIS3, ade2::GAL2-ADE2, met2::GAL7-lacZ. Uetz et al. 2000).

9.2: Random Mutagenesis

Randomly mutated CDK A;1 coding sequences were produced using error-prone PCR. 30 ng of pAD-cdc2-1 plasmid were added to a PCR mix (dATP 0.2 mM, dGTP 0.2 mM, dCTP 1 mM, dTTP 1 mM, buffer 1×, MnCl2 0.5 mM, forward primer (AGGGATGTTTAATACCACTAC) 1 mM, reverse primer (GCACAGTTGAAGTGAACTTGC) 1 mM, Taq polymerase 1 U). The reaction mixture was denatured for 5 minutes at 94° C., 30 cycles of 1 minute denaturing at 94° C., 1 minute annealing at 40° C., 2 minutes elongation at 72° C., followed by a last elongation of 5 minutes at 72° C. This procedure introduces errors at a rate of 2-3 base substitutions per kilo base pair (Miyazaki and Arnold (1999). J Mol Evol 49:716-720; Shafikhani et al (1997). BioTechniques 23:304-310). The PCR fragment was cloned directly by gap-repair cloning (Fusco et al., (1999) Yeast 15: 715-720) into pAD vector linearized by EcoR I and Sal I digestion, using the yeast strain MaV203 (Life technology) containing the pBD-OsICK2 or pBD-OsICK4 plasmid as recipient. CDK A;1 was selected by reverse-two-hybrid methods. The yeast strains were plated on -Leu -Trp selective medium containing 0.2% (w/v) 5-fluoro-orotic acid and incubated at 28° C. Yeast colonies able to survive on such a medium were CDK A;1 mutants unable to interact with ICK2 or ICK4.

9.3: Two-Hybrid Mating Procedure

Yeast strains PJ69-4A (MAT-a) and PJ69-4alpha (MAT-alpha) were transformed either with pAD or pBD plasmid containing the coding sequences of interest. Individual strains were plated in strips, either horizontally or vertically, respectively on -Leu or -Trp selective medium and were grown at 28° C. for 1-2 days. The yeast strips were transferred onto non-selective YPG medium in such a way that the strips formed a grid, with AD and BD strains mixing at strip intersections. The yeast was incubated for 8 hours at 28° C. to allow mating between MAT-a and MAT-alpha strains to occur. The grid was subsequently transferred on -Leu -Trp -H is -Ade selective medium and incubated at 28° C. for 2 days. Yeast able to grow on such a medium were MAT-a/alpha diploid yeasts containing AD and BD plasmid expressing interacting proteins (James et al. (1996) Genetics 144: 1425-1436. Uetz et al. (2000) Nature 403: 623-627).

9.4: Yeast Expression Vectors Construction and Mutant Complementation

Yeast galactose-inducible expression vector pESC-Trp (Stratagene) was cut with Apa I and Sal I, ligated to the Apa I-Sal I fragment from pBSK-GWA, which contains the Gateway cassette A cloned at the EcoR V site of pBlueScript. The resulting vectors, pE-GW, was introduced into E. coli DB3.1, and contained the AttR1 site of the gateway cassette directly downstream of the GAL1 promoter. The coding sequences of interest were transferred into the pE-GW vector from the pDONR entry clone via the gateway LR reaction.

Yeast strain US102 (Loy C J et al. (1999) Mol Cell Biol. 19: 3312-3327) was mutated in the CDK cdc28, and is unable to grow at 37° C., but able to grow at 24° C. Transgenic yeast strain containing plasmids for expression of interesting genes were grown on galactose-containing medium, at 37° C. The yeast strains expressing genes able to rescue the cdc28 mutation are those able to grow at that restrictive temperature.

### Results

9.5: Screening

Rice CDK A;1 mutant library in pAD vector was generated by error-prone PCR and gap repair cloning in the MaV203 strain as described. The library was screened against rice ICK4 by reverse two-hybrid. About 100 000 mutants were screened, and 79 colonies able to grow on 5-fluoroorotic acid were obtained. Among them, 74 were transferred into PJ69-4A for further analysis by mating two-hybrid. The mutant coding sequences were then sequenced. An average mutagenicity level of 5.6±3.4 substitutions per kilo base pair was observed.

9.6: Characterization

Wild-type rice cdc2-1 is able to bind strongly to the rice ICK4 and the mouse herpesvirus Cyclin D homologue and less strongly the rice cyclin D3. The capacity of the cdc2 mutants to bind these 3 proteins was investigated. Their capacity to complement the US102 cdc28 yeast mutant strain, like the wild type cdc2 protein, was also investigated.

9.6.1: Mutants that Bind to Cyclin, but not to ICKs

Mutants of particular interest were those able to bind cyclins, but not ICK4, whilst still retaining their capacity to complement the yeast mutant. These mutants render a cyclin-CDK complex insensitive to ICK-mediated inhibition. Three mutants of particular interest identified are shown in Table A repeated below.

TABLE A
Mutants that bind to cyclin, but not to ICKs

| Mutant | SEQ ID NO | Mutation Position | | |
|---|---|---|---|---|
| 1 | SEQ ID NO: 9 | Y4H | V79D | A152T |
| 2 | SEQ ID NO: 10 | I30T | | |
| 3 | SEQ ID NO: 11 | E5V | R122S | K143E |

Mutations positions are calculated from the first methionine of CDK A;1.

9.6.2: Mutants that Bind CKI but not Cyclin

Also interesting are those mutants able to bind ICK, but not cyclins, and which have lost their capacity to complement the yeast US102 mutant. Such mutants would titrate out the ICKs rendering them unable to inhibit the CDK-cyclin complex. The mutants shown in Table B repeated below show such characteristics.

TABLE B
Mutants that bind CKI but not cyclin

| Mutant | SEQ ID NO | Mutant Position | | | | | | |
|---|---|---|---|---|---|---|---|---|
| 4 | SEQ ID NO: 12 | G154R | | | | | | |
| 5 | SEQ ID NO: 13 | Q3P | E38A | R137L | S182A | I193T | M267V | R279Q |

1. Method for improving plant growth characteristics selected from one or more of increased yield, increased growth rate and modified architecture, said method comprising increasing expression in a plant of a nucleic acid encoding a B-type CDK protein and/or increasing activity and/or levels in a plant of a B-type CDK protein.

2. Method according to claim 1, wherein said increasing expression is effected by introducing and expressing in a plant a B-type CDK nucleic acid.

3. Method according to claim 1 or 2, wherein said B-type CDK is derived from a plant, algal or fungal source.

4. Method according to claim 3, wherein said B-type CDK derived from a plant is preferably from a dicotyledonous plant, further preferably from the family Brassicaceae, more preferably the nucleic acid sequence is from Arabidopsis thaliana.

5. Method according to any claim 3, wherein said B-type CDK is a class 1 B-type CDK, preferably a CDK B1;1 from Arabidopsis thaliana or a CDK B1;2 from Arabidopsis thaliana.

6. Method according to claim 5, wherein said B-type CDK is a class 2 B-type CDK, preferably a CDK B2;2 from Arabidopsis thaliana.

7. Method according to claim 5, wherein said CDK B1;1 nucleic acid is as represented by SEQ ID NO: 1 or by a portion thereof, or by a nucleic acid sequence capable of hybridising therewith, and wherein the CDK B1; 1 protein is as represented by SEQ ID NO: 2, or a homologue, derivative or active fragment thereof.

8. Method according to claim 5, wherein said CDK B1;2 nucleic acid is as represented by SEQ ID NO: 3 or by a portion thereof, or by a nucleic acid sequence capable of hybridising therewith, and wherein the CDK B1;2 protein is as represented by SEQ ID NO: 4, or a homologue, derivative or active fragment thereof.

9. Method according to claim 6, wherein said CDK B2;2 nucleic acid is as represented by SEQ ID NO: 5 or by a portion thereof, or by a nucleic acid sequence capable of hybridising therewith, and wherein the CDK B2;2 protein is as represented by SEQ ID NO: 6, or a homologue, derivative or active fragment thereof.

10. Method according to claim 4, wherein said B-type CDK is a variant nucleic acid or variant amino acids selected from:
(a) Functional portions of a B-type CDK nucleic acid/gene;
(b) Sequences capable of hybridising to a B-type CDK nucleic acid/gene;
(c) Alternative splice variants of a B-type CDK nucleic acid/gene;
(d) Allelic variants of a B-type CDK nucleic acid/gene;
(e) Homologues, derivatives and active fragments of a B-type CDK protein;
(f) Mutant B-type CDKs;

11. Method according to claim 5, wherein expression of said CDK B1;1 nucleic acid is driven by a promoter active in young, expanding tissue, preferably wherein said promoter is a beta expansin promoter.

12. Method according to claim 5, wherein expression of said CDK B1;2 nucleic acid and wherein expression of said CDK B2;2 nucleic acid is driven by a constitutive promoter, preferably wherein said promoter is a GOS 2 promoter.

13. Method according to claim 1, wherein said increased yield comprises one or more of: an increase in area, an increase in the number of panicles, an increase in height, an increase in the number of seeds, an increase in the number of filled seeds, an increase in the total weight of seeds, an increase in thousand kernel weight (TKW) and an increase in harvest index, each relative to control plants.

14. Method according to claim 1, wherein said modified architecture includes one or more of: increase in aboveground area, increase in the number of panicles and an increase in height.

15. Plants obtainable by a method according to claim 6.

16. Construct comprising:
(a) a B-type CDK gene/nucleic acid encoding a B-type CDK protein; or
(b) a nucleic acid encoding a CDK mutant, which CDK mutant comprises at least one of the seven amino acid position changes shown in Table A or at least one of the eight amino acid position changes shown in Table B;
(c) one or more control sequences capable of driving expression of the nucleic acid of (a) or (b); and optionally
(d) a transcription termination sequence.

17. Construct according to claim 16, wherein said nucleic acid of (a) is a CDK B1;2 nucleic acid as represented by SEQ ID NO: 3 or by a portion thereof, or by a nucleic acid sequence capable of hybridising therewith, which nucleic acid encodes a CDK B1;2 protein as represented by SEQ ID NO: 4, or a homologue, derivative or active fragment thereof.

18. Construct according to claim 16, wherein said nucleic acid of (a) is a CDK B2;2 nucleic acid as represented by SEQ ID NO: 5 or by a portion thereof, or by a nucleic acid sequence capable of hybridising therewith, which nucleic acid encodes a CDK B2;2 protein as represented by SEQ ID NO: 6, or a homologue, derivative or active fragment thereof.

19. Construct according to claim 16, wherein said control sequence comprises a constitutive promoter, preferably a GOS 2 promoter.

20. Method for the production of a transgenic plant having improved growth characteristics selected from any one or more of: increased yield, increased growth rate and modified architecture, which growth characteristics are improved relative to growth characteristics of corresponding wild type plants, said method comprising the steps of:
(a) introducing into a plant or a plant cell a B-type CDK gene/nucleic; or
(b) a nucleic acid encoding a CDK mutant, which CDK mutant comprises at least one of the seven amino acid position changes shown in Table A;
(c) cultivating the plant cell under conditions promoting regeneration and mature plant growth.

21. Transgenic plant having improved growth characteristics selected from any one or more of: increased yield, increased growth rate and modified architecture, characterised in that said plant has increased expression of a B-type CDK nucleic acid and/or increased activity and/or levels in a plant of a B-type CDK protein relative to corresponding wild type plants.

22. Transgenic plant according to claim 21, wherein said plant is a monocotyledonous plant.

23. (canceled)

24. A composition comprising a protein represented by SEQ ID NO 2, SEQ ID NO: 4 or SEQ ID NO: 6 or a homologue, derivative, or active fragment of any of the aforementioned sequences, for use as a growth regulator.

25. Screening method for the identification of mutant plant cyclin dependent kinases (CDKs) having enhanced CDK activity relative to corresponding non-mutated plant CDKs, which method comprises the steps of:
(a) Providing plant-derived CDK mutants;
(b) Identifying cyclin dependent kinase inhibitor (ICK) non-reacting mutants;
(c) Identifying mutants having cyclin-binding activity; and optionally followed by,
(d) A yeast complementation assay on resultant mutants from steps (b) and (c).

26. Screening method for the identification of substantially non-active plant CDKs, but which are capable of binding to plant ICKs, comprising the steps of:
(a) Providing plant-derived CDK mutants;
(b) Identifying plant-derived ICK binding mutants; and
(c) Identifying non-cyclin-binding mutants.

27. Screening method according to claim 25 or 26 wherein said CDK mutants are provided by the steps of:
(a) Providing a wild-type Plant CDK; and
(b) Mutating said CDK at least at 1 amino-acid position;

28. Mutant CDKs obtainable by the method according to claim 27, which mutant CDKs are indicated in Table A.

29. Mutant CDKs obtainable by the method according to claim 27, which mutant CDKs are indicated in Table B.

30. An isolated nucleic acid molecule comprising:
(a) a nucleic acid encoding a CDK mutant represented by any one of SEQ ID NO: 9, SEQ ID NO: 10, SEQ ID NO: 11, SEQ ID NO: 12 and SEQ ID NO: 13;
(b) a nucleic acid encoding a homologue, derivative or active fragment of a CDK mutant represented by any one of SEQ ID NO: 9, SEQ ID NO: 10, SEQ ID NO: 11, SEQ ID NO: 12 and SEQ ID NO: 13, which homologue, derivative or active fragment comprises at least one of the seven amino acid position changes shown in Table A or at least one of the eight amino acid position changes shown in Table B;
(c) a nucleic acid capable of hybridising with a nucleic acid of (a) or (b) above, wherein the hybridising sequence encodes an amino acid comprising at least one of the seven amino acid position changes shown in Table A or at least one of the eight amino acid position changes shown in Table B;
(d) a nucleic acid of (a) to (c) above which is degenerate as a result of the genetic code;
(e) allelic variants of the nucleic acids of (a) to (d), which allelic variant encodes an amino acid comprising at least one of the seven amino acid position changes shown in Table A or at least one of the eight amino acid position changes shown in Table B; and
(f) alternative splice variants of the nucleic acids of (a) to (e), which alternative splice variants encode an amino acid comprising at least one of the seven amino acid position changes shown in Table A or at least one of the eight amino acid position changes shown in Table B.

31. A CDK mutant, comprising:
(a) an amino acid represented by any one of SEQ ID NO: 9, SEQ ID NO: 10, SEQ ID NO: 11, SEQ ID NO: 12 and SEQ ID NO: 13; and
(b) a fragment of an amino acid represented by any one of SEQ ID NO: 9, SEQ ID NO: 10, SEQ ID NO: 11, SEQ ID NO: 12 and SEQ ID NO: 13, which fragment comprises at least seven amino acid position changes shown in Table A or at least one of the eight amino acid position changes shown in Table B.
